(19) United States
(12) Patent Application Publication
(10) Pub. No.: US 2017/0266197 A1
(43) Pub. Date: Sep. 21, 2017

(54) METHODS AND COMPOSITIONS FOR TREATING HEPATITIS B VIRUS INFECTIONS

(71) Applicants: Indiana University Research and Technology Corporation, Indianapolis, IN (US); Assembly Biosciences, Inc., Carmel, IN (US)

(72) Inventors: Adam Zlotnick, Bloomington, IN (US); William W. Turner, Bloomington, IN (US); Lee Daniel Arnold, Bloomington, IN (US)

(73) Assignees: Indiana University Research and Technology Corporation, Indianapolis, IN (US); Assembly Biosciences, Inc., Carmel, IN (US)

(21) Appl. No.: 15/310,070

(22) PCT Filed: May 11, 2015
(86) PCT No.: PCT/US2015/030064

§ 371 Date: Nov. 9, 2016

Related U.S. Application Data
(60) Provisional application No. 61/990,801, filed on May 9, 2014.

(51) Int. Cl. A61K 31/5377; A61K 31/506; G01N 33/50; A61K 31/445; G01N 33/576; A61K 45/06; A61K 31/18

(52) U.S. Cl. CPC A61K 31/5377; A61K 45/06; A61K 31/506; A61K 31/18; A61K 31/445; G01N 33/5762; G01N 33/5023; G01N 33/5032; G01N 2500/04

(57) ABSTRACT

Described herein are methods for identifying compounds useful for the treatment of infection by hepatitis B virus (HBV) and for identifying compounds useful for the same.

### CROSS-REFERENCE TO RELATED APPLICATIONS

This application claims the benefit of and priority to co-pending U.S. Provisional Patent Application No. 61/990,801, filed May 9, 2014, the entire contents of which are incorporated by reference herein.

### STATEMENT OF GOVERNMENT SUPPORT

This invention was made with government support under AI067417 and AI077688 awarded by the National Institutes of Health. The Government has certain rights in the invention.

### TECHNICAL FIELD

The present disclosure relates to methods for treating hepatitis B virus (HBV) infections and for identifying compounds useful for the same.

### BACKGROUND

The hepatitis B virus (HBV), which belongs to the hepadnavirus family, is a causative agent of acute and chronic hepatitis. HBV infections are the world's ninth leading cause of death. HBV infection often leads to acute hepatitis and liver damage, and causes abdominal pain, jaundice, and elevated blood levels of certain enzymes. HBV can cause fulminant hepatitis, a rapidly progressive form of the disease in which massive sections of the liver are destroyed. Many patients recover from acute viral hepatitis, but in certain other patients, especially young children, viral infection persists for an extended, or indefinite, period, causing a chronic infection. Chronic infections can lead to chronic persistent hepatitis. Chronic persistent hepatitis can cause fatigue, liver damage, cirrhosis of the liver, and hepatocellular carcinoma, a primary liver cancer.

HBV infection is a serious problem among the homo- and heterosexual population, intravenous drug users, organ transplant recipients, and blood transfusion patients. New infection with HBV can be prevented by vaccination. However, the present vaccination is not relevant for the approximately 350 million chronic carriers worldwide.

It has been observed that suppression or eradication of the replication of HBV in the liver leads to improved liver pathology and decreased progression to liver cirrhosis and hepatocellular carcinoma.

One of the current therapies approved in the United States for treating chronic hepatitis B infection is alpha interferon, which is far from ideal. According to the American Liver Foundation and the International Hepatitis Foundation, patients with conditions such as advanced hepatitis, HIV co-infection, drug abuse or others are not eligible for this treatment, resulting in less than 50% of chronic carriers obtaining this therapy. Of these patients, only about 40% respond to the treatment. Many of these patients also relapse after treatment is stopped, and only about 30% of the patients show a long term benefit. Viral disappearance is only seen in about 10-20% of the treated patients. These data suggest that there is an extremely low response rate in patients treated with alpha interferon. In addition to the low response rate, interferon therapy causes severe side effects such as insomnia, depression, nausea, vomiting, fever and fatigue. Another approved class of drugs for treating HBV infection is reverse transcriptase inhibitors exemplified by lamivudine, entecavir, and tenofovir. Although reverse transcriptase inhibitors have good antiviral activity, resistance can develop during treatment, there is cross-reactivity of resistance, and side effects such as kidney damage. There is also cross-reactivity between reverse transcriptase inhibitors for HBV and HIV. Furthermore, extended years of therapy with reverse transcriptase inhibitors rarely results in a cure. Discontinuation of therapy leads to risk of rebound, which can be life threatening.

The development of novel therapies for HBV infection requires new antivirals that block viral life cycle functions other than those associated with the viral polymerase. Core proteins (Cp) have been shown to interact with histones and to bind the nuclear cccDNA, possibly contributing to the regulation of cccDNA function and the maintenance of the cccDNA stability (Bock, J M B 2001; Pollicino, Gastroenterology 2006; Guo, Epigenetics 2011; Belloni et al., Digestive and Liver Disease 2015). Accordingly, there is a need in the art for compounds that modulate cccDNA regulation by Cp, thereby providing antiviral activity against HBV.

### SUMMARY OF THE INVENTION

Described herein is the discovery that certain compounds that allosterically or orthosterically modify Cp (referred to herein as Cp assembly modifiers (CpAMs)), can affect assembly of Cp as well as exhibit non-assembly effects useful in treating or clinically curing a patient infected by HBV. As used herein, assembly can mean formation of small Cp oligomers and/or assembly of the entire capsid. For example, CpAMs (e.g., HAP12) can both drive Cp assembly and modulate direct or indirect interactions between core protein and DNA (e.g., cccDNA), thereby disrupting the HBV life cycle and providing antiviral activity against HBV. Furthermore, targeting HBV by both (1) modulating Cp assembly and (2) modulating direct or indirect interactions between core protein and DNA (e.g., cccDNA) can result in an improved clinical outcome compared to administration of either compound alone.

In addition, certain CpAMs (e.g. hetero-aryl-dihydropyrimidines (HAPs) such as HAP12, as well as the compounds AT130, GLS4 (HecPharm), and N890 (Novira)) enhance the rate and the extent of Cp assembly over a broad concentration range and act as allosteric effectors to induce an assembly-active state or, at high concentration, stabilize preferentially non-capsid polymers of Cp interfering with normal virion assembly, resulting in an antiviral effect by inhibiting HBV replication. (Deres, Science 2003; Stray, PNAS 2005).

Without wishing to be bound by theory, these small molecules make reactions proceed faster and increase oligomerization relative to control (no drug) assembly reactions. To induce assembly, a CpAM need only interact with a small number of subunits to form a nucleus; nucleation is typically the slow step in assembly (Zlotnick et al. 1999; Endres and Zlotnick 2003; Katen and Zlotnick 2009). Supporting nucleation, by itself, can increase assembly kinetics. However, to increase oligomerization, the CpAM must strengthen the average association energy between subunits (i.e. make the association energy more negative). This cannot be accomplished by having one CpAM bind to a capsid; rather it is accomplished by CpAMs binding to subunits resulting in a dose dependent increase in capsid stability (Bourne et al., 2008). Thus a CpAM that increases the amount of capsid formed, necessarily thermodynamically stabilizes capsid and necessarily binds capsid. A corollary to this effect is that capsids are a sink for free Cp dimers. That is, some CpAMs, like the HAPs, will bind capsid with stronger affinity than they bind free Cp dimer. In other words, a small concentration of assembly-inducing CpAMs may be able to nucleate assembly, but in order to maximize the amount of Cp assembled to deplete free dimer, fill binding sites available in capsid and non-capsid polymer, and have CpAM available to bind free Cp dimer, a much higher concentration of CpAM is required. In addition, an assembly-inducing CpAM is expected to bind Cp dimer with weaker affinity than it binds capsid. By binding to Cp, the CpAM can affect Cp activities outlined above, other than assembly.

Described herein are methods for treating or clinically curing a patient infected by hepatitis B virus (HBV), including administering to the patient a therapeutically effective amount of a compound capable of modulating core protein-mediated regulation of DNA (e.g., cccDNA) in an HBV infected cell of the patient. In some embodiments, the compound is also capable of modulating core protein assembly. The method can also comprise administering to the patient a therapeutically effective amount of a compound capable of modulating core protein assembly. In some embodiments, administration of the two compounds results in an improved clinical outcome compared to administration of either compound alone. In other embodiments, administration of the two compounds results in a synergistically improved clinical outcome compared to administration of either compound alone.

Also described herein are methods for treating or clinically curing a patient infected by hepatitis B virus (HBV), the method comprising the step of administering to the patient a compound capable of modulating core protein assembly, wherein the compound is administered at a dose sufficient to modulate core protein-mediated regulation of DNA (e.g., cccDNA). In some embodiments, administration of the compound alters HBsAg, HBeAg, or viral RNA levels. In certain embodiments the compound is capable of (a) modulating the structure of core protein; (b) modulating the function of core protein; (c) modulating the binding of core protein to DNA (e.g., cccDNA); (d) depleting the amount of free core protein dimer available to bind to DNA (e.g., cccDNA); (e) altering nuclear import or export of core protein; (f) altering an interaction between cccDNA and a chromatin component; (g) altering an interaction between core protein and a chromatin component; (h) altering the rate, quantity, quality or stability of RNA expressed from DNA (e.g., cccDNA); (i) altering the stability or maintenance of DNA (e.g., cccDNA); and/or (j) modulating an innate immune response against HBV. In certain embodiments, the compound acts allosterically or orthosterically.

In some embodiments, the method also includes administering an additional compound. For example, one or more of the following compounds can be administered in combination with the compounds described herein: a nucleoside HBV polymerase inhibitor, a nucleotide HBV polymerase inhibitor, a modified nucleic acid, a peptide entry inhibitor, an interferon (Type I, II or III), a lymphotoxin beta agonist, a Toll-like receptor agonist, a non-nucleoside small molecule HBV polymerase inhibitor, a non-nucleotide small molecule HBV polymerase inhibitor, a compound affecting capsid maturation, an HBV cccDNA transcriptional modulator, a cccDNA biosynthesis inhibitor, a subviral particle secretion inhibitor, a checkpoint modulator, an siRNA, a therapeutic vaccine, an entry inhibitor, a transcriptional modifier, a topoisomerase inhibitor, a compound that modulates presentation of HBV antigen via MHC, an HBV RNase H inhibitor, a proteasome inhibitor, a cyclophilin inhibitor, a transcription activator-like effector nuclease (TALEN), a DNA cleavage enzyme such as CAS9/CRISPR targeting HBV cccDNA, a dominant negative HBV mutant, a second mitochondrial-derived activator of caspases (SMAC) mimetic, nucleic acid-based polymer (NAP) such as REP-2139Ca and REP-2055, a Stimulator of Interferon Genes (STING) such as DMXAA and 2′3′-cGAMP, and an inhibitory peptide. Checkpoint modulators include an anti-PD-1 antibody, an anti-PD-L1 antibody, or an anti-CTLA4 antibody. In some embodiments, the siRNA targets HBV (e.g., ISIS-HBV mRNA-targeted antisense) or host RNA. SMAC mimetics include birinapant (TL32711), LCL161 (Novartis), GDC-0917 (Genentech), HGS1029 (Human Genome Sciences), and AT-406 (Ascenta).

The methods described herein can include administration of any one of the following compounds:

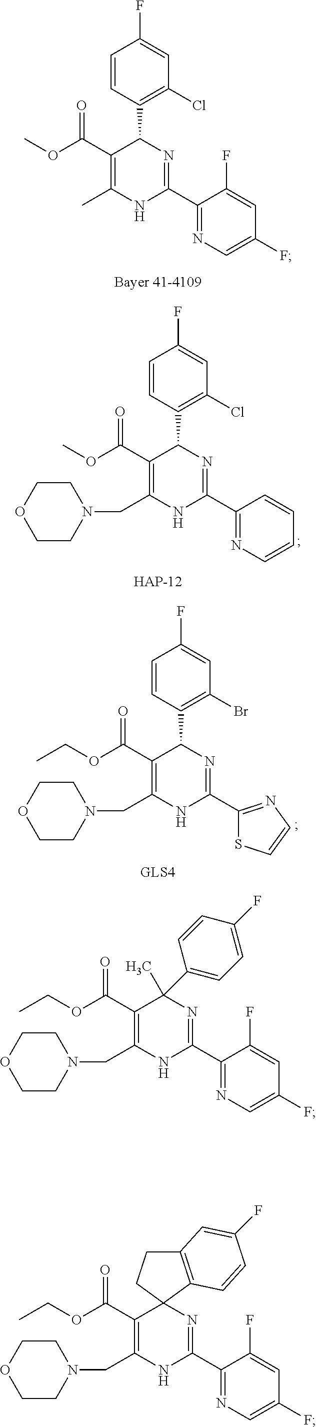

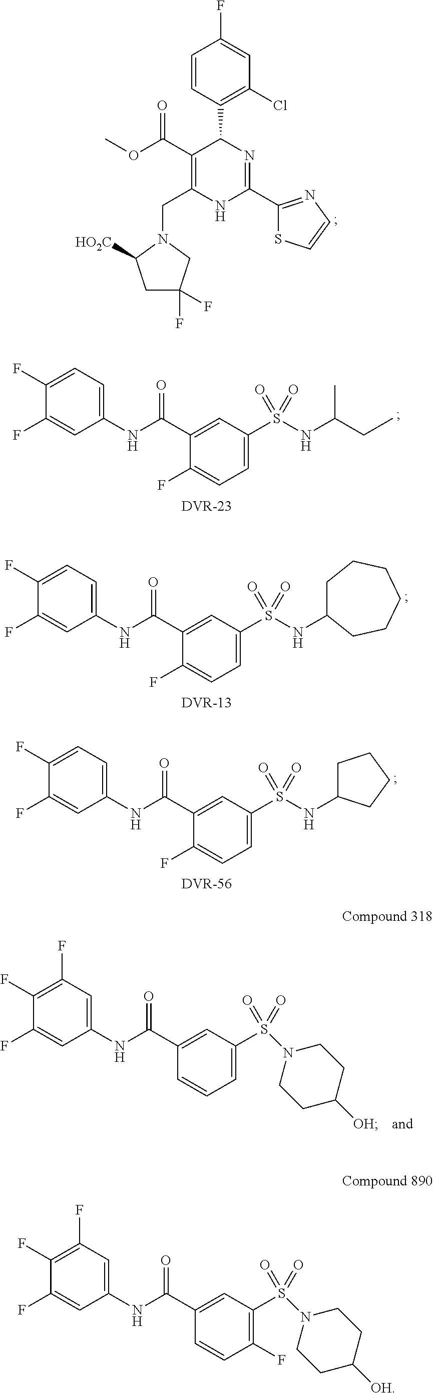

The methods described herein can include administration of any of the capsid promoting and HBsAg reducing molecules described in International Publication No. WO/2015/057945, U.S. Provisional Patent Application No. 62/148,994, and in International Patent Application No. PCT/US2015/020444.

For example, the methods described herein can include administration of a compound of Formula 1 (from International Publication No. WO/2015/057945) having the structure:

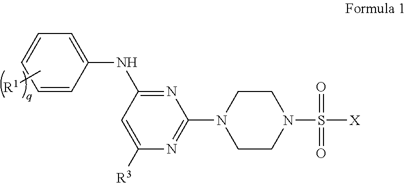

or a pharmaceutically acceptable salt or stereoisomer thereof, wherein:
X is selected from the group consisting of

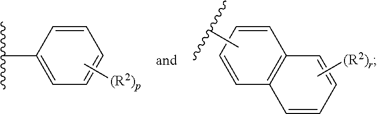

q is 0, 1, 2, 3 or 4;
p is 0, 1, 2, 3, or 4;
r is 0, 1, 2, 3, or 4;
R¹ is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″);
w is 0, 1 or 2;
R′ is independently for each occurrence selected from the group consisting of —H and —C₁-C₆alkyl;
R″ is independently for each occurrence selected from the group consisting of —H and —C₁-C₆alkyl; or R′ and R″ are taken together with the nitrogen atom to which they are attached to form a 4-7 membered heterocyclic ring;
R² is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″); and
R³ is selected from the group consisting of —H and —C₁-C₆alkyl;
wherein C₁-C₆alkyl or C₁-C₆alkoxy may be independently for each occurrence optionally substituted with one, two, or three halogens.

The methods described herein can also include a compound of Formula 1 (described in U.S. Provisional Patent Application No. 62/148,994) having the structure:

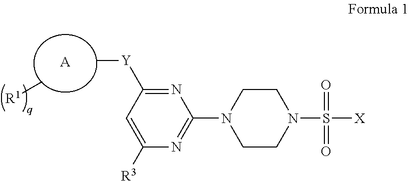

or a pharmaceutically acceptable salt or stereoisomer thereof, wherein:

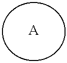

is selected from the group consisting of phenyl, naphthyl, and heteroaryl;
Y is selected from the group consisting of a bond, —O—, —S(O)_{w}—, and —N(R′)—;
X is selected from the group consisting of phenyl, naphthyl, and heteroaryl; wherein X
is optionally substituted with one, two, three, or four R² groups;
provided that at least one of

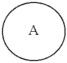

or X is a heteroaryl;
R¹ is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″);
q is 0, 1, 2, 3 or 4;
w is 0, 1 or 2;
R′ is independently for each occurrence selected from the group consisting of —H and —C₁-C₆alkyl;
R″ is independently for each occurrence selected from the group consisting of —H and —C₁-C₆alkyl; or R′ and R″ are taken together with the nitrogen atom to which they are attached to form a 4-7 membered heterocyclic or heteroaryl ring, each of which is optionally substituted with an oxo group;
R² is independently for each occurrence selected from the group consisting of —H, —C₁-C₆alkyl, —C₁-C₆alkoxy, —C₁-C₆alkyl-O—C₁-C₆alkyl, halogen, oxo, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″); and
R³ is selected from the group consisting of —H, —C₁-C₆alkyl, —N(R′)(R″), —N(R′)C₁-C₆alkyl-N(R′)(R″), —OH, —C₁-C₆alkoxy, —O—C₁-C₆alkyl-OR′, —O-heterocyclyl, —O-heteroaryl, —O—C₁-C₆alkyl-heteroaryl, —C₁-C₆alkyl-heteroaryl, heterocyclyl, and heteroaryl, wherein heterocyclyl and heteroaryl are optionally substituted with one or two C₁-C₆alkyl or halogen;
wherein C₁-C₆alkyl or C₁-C₆alkoxy may be independently for each occurrence optionally substituted with one, two, or three halogens.

The methods described herein can also include a compound of Formula 2 (described in U.S. Provisional Patent Application No. 62/148,994) having the structure:

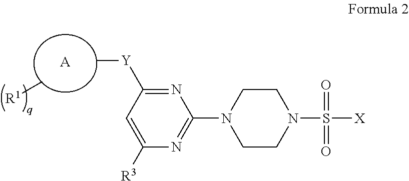

or a pharmaceutically acceptable salt or stereoisomer thereof, wherein:

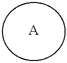

is selected from the group consisting of phenyl, naphthyl, and heteroaryl;
Y is selected from the group consisting of a bond, —O—, and —S(O)_{w}—;
X is selected from the group consisting of phenyl, naphthyl, and heteroaryl; wherein X
is optionally substituted with one, two, three, or four R² groups;
R¹ is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″);
q is 0, 1, 2, 3 or 4;
w is 0, 1 or 2;
R′ is independently for each occurrence selected from the group consisting of —H and —C₁-C₆alkyl;
R″ is independently for each occurrence selected from the group consisting of —H and —C₁-C₆alkyl; or R′ and R″ are taken together with the nitrogen atom to which they are attached to form a 4-7 membered heterocyclic or heteroaryl ring, each of which is optionally substituted with an oxo group;
R² is independently for each occurrence selected from the group consisting of —H, —C₁-C₆alkyl, —C₁-C₆alkoxy, —C₁-C₆alkyl-O—C₁-C₆alkyl, halogen, oxo, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″); and
R³ is selected from the group consisting of —H, —C₁-C₆alkyl, —N(R′)(R″), —N(R′)C₁-C₆alkyl-N(R′)(R″), —N(R′)—C₁-C₆alkyl-OR′, —OH, —C₁-C₆alkoxy, —O—C₁-C₆alkyl-OR′, —O-heterocyclyl, —O-heteroaryl, —O—C₁-C₆alkyl-heteroaryl, —C₁-C₆alkyl-heteroaryl, heterocyclyl, and heteroaryl, wherein heterocyclyl and heteroaryl are optionally substituted with one or two C₁-C₆alkyl or halogen;
wherein C₁-C₆alkyl or C₁-C₆alkoxy may be independently for each occurrence optionally substituted with one, two, or three halogens.

The methods described herein can also include a compound of Formula 3 (described in U.S. Provisional Patent Application No. 62/148,994) having the structure:

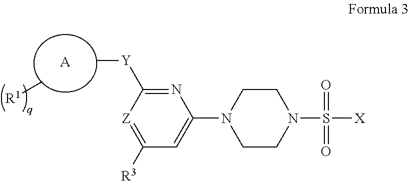

or a pharmaceutically acceptable salt or stereoisomer thereof, wherein:

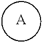

is selected from the group consisting of phenyl, naphthyl, and heteroaryl;
Y is selected from the group consisting of a bond, —O—, —S(O)_{w}—, and —N(R′)—;
Z is CH or N;
X is selected from the group consisting of phenyl, naphthyl, and heteroaryl; wherein X is optionally substituted with one, two, three, or four R² groups;
R¹ is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″);
q is 0, 1, 2, 3 or 4;
w is 0, 1 or 2;
R′ is independently for each occurrence selected from the group consisting of —H and —C₁-C₆alkyl;
R″ is independently for each occurrence selected from the group consisting of —H and —C₁-C₆alkyl; or R′ and R″ are taken together with the nitrogen atom to which they are attached to form a 4-7 membered heterocyclic or heteroaryl ring, each of which is optionally substituted with an oxo group;
R² is independently for each occurrence selected from the group consisting of —H, —C₁-C₆alkyl, —C₁-C₆alkoxy, —C₁-C₆alkyl-O—C₁-C₆alkyl, halogen, oxo, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″); and
R³ is selected from the group consisting of —H, —C₁-C₆alkyl, —N(R′)(R″), —N(R′)C₁-C₆alkyl-N(R′)(R″), —N(R′)—C₁-C₆alkyl-OR′, —OH, —C₁-C₆alkoxy, —O—C₁-C₆alkyl-OR′, —O-heterocyclyl, —O-heteroaryl, —O—C₁-C₆alkyl-heteroaryl, —C₁-C₆alkyl-heteroaryl, heterocyclyl, and heteroaryl, wherein heterocyclyl and heteroaryl are optionally substituted with one or two C₁-C₆alkyl or halogen;
wherein C₁-C₆alkyl or C₁-C₆alkoxy may be independently for each occurrence optionally substituted with one, two, or three halogens.

The methods described herein can also include a compound of Formula 4 (described in U.S. Provisional Patent Application No. 62/148,994) having the structure:

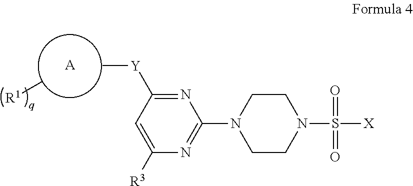

or a pharmaceutically acceptable salt or stereoisomer thereof, wherein:

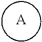

is selected from the group consisting of phenyl, naphthyl, and heteroaryl;
Y is selected from the group consisting of a bond, —O—, —S(O)_{w}—, and —N(R′)—;
X is selected from the group consisting of phenyl, naphthyl, and heteroaryl; wherein X is optionally substituted with one, two, three, or four R² groups;
R¹ is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″);
q is 0, 1, 2, 3 or 4;
w is 0, 1 or 2;
R′ is independently for each occurrence selected from the group consisting of —H and —C₁-C₆alkyl;
R″ is independently for each occurrence selected from the group consisting of —H and —C₁-C₆alkyl; or R′ and R″ are taken together with the nitrogen atom to which they are attached to form a 4-7 membered heterocyclic or heteroaryl ring, each of which is optionally substituted with an oxo group;
R² is independently for each occurrence selected from the group consisting of —H, —C₁-C₆alkyl, —C₁-C₆alkoxy, —C₁-C₆alkyl-O—C₁-C₆alkyl, halogen, oxo, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″); and
R³ is selected from the group consisting of —N(R′)(R″), —N(R)C₁-C₆alkyl-N(R′)(R″), —N(R)—C₁-C₆alkyl-OR′, —OH, —C₁-C₆alkoxy, —O—C₁-C₆alkyl-OR′, —O-heterocyclyl, —O-heteroaryl, —O—C₁-C₆alkyl-heteroaryl, —C₁-C₆alkyl-heteroaryl, heterocyclyl, and heteroaryl, wherein heterocyclyl and heteroaryl are optionally substituted with one or two C₁-C₆alkyl or halogen;
wherein C₁-C₆alkyl or C₁-C₆alkoxy may be independently for each occurrence optionally substituted with one, two, or three halogens

The methods described herein can also include a compound of Formula 4 (described in International Patent Application No. PCT/US2015/020444) having the structure:

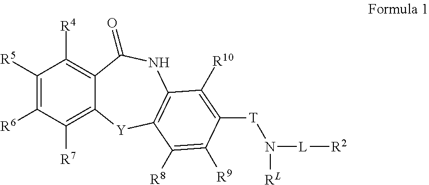

wherein:

T is selected from the group consisting of —C(O)—, —CH₂—C(O)—, —N(C(O)—CH₃)—, —NH—, —O—, and —S(O)_{z}—, where z is 0, 1 or 2;

Y is C(R¹¹)₂, S(O)_{y}, NR_{Y} and O wherein y is 0, 1, or 2;

R_{Y} is selected from the group consisting of H, methyl, ethyl, propyl, phenyl and benzyl;

R_{L} is selected from the group consisting of H, methyl, and —C(O)—C₁₋₃alkyl;

L is a bond or C₁₋₄ straight chain alkylene optionally substituted by one or two substituents each independently selected from the group consisting of methyl (optionally substituted by halogen or hydroxyl), ethenyl, hydroxyl, NR′R″, phenyl, heterocycle, and halogen and wherein the C₁₋₄ straight chain alkylene may be interrupted by an —O—;

R² is selected from the group consisting of H,

phenyl or naphthyl (wherein the phenyl or naphthyl may be optionally substituted with one, two, three or more substituents selected from the group consisting of halogen, hydroxyl, nitro, cyano, carboxy, C₁₋₆alkyl, C₂₋₆alkenyl, C₂₋₆alkynyl, C₁₋₆alkoxy, NR′R″, —C(O)—NR′R″, —C(O)—C₁₋₆alkyl, —C(O)—C₁₋₆alkoxy, phenyl (optionally substituted by one, two or three substituents each independently selected from the group consisting of halogen, hydroxyl, cyano, C₁₋₆alkyl, C₂₋₆alkenyl, C₂₋₆alkynyl, C₁₋₆alkoxy, NR′R″, C(O)—NR′R″, —C(O)—C₁₋₆alkyl, —C(O)—C₁₋₆alkoxy, —S(O)_{w}—C₁₋₆alkyl (where w is 1, 2 or 3), S(O)_{w}—NR′R″ (where w is 1, 2 or 3), —NR′—S(O)_{w}, and —S(O)_{w}—NR′R″ (where w is 1, 2 or 3)), heteroaryl (optionally substituted by one, two or three substituents each independently selected from the group consisting of halogen, hydroxyl, C₁₋₆alkyl, C₂₋₆alkenyl, C₂₋₆alkynyl, C₁₋₆alkoxy, NR′R″, C(O)—NR′R″, —C(O)—C₁₋₆alkyl, —C(O)—C₁₋₆alkoxy, —S(O)_{w}—C₁₋₆alkyl (where w is 1, 2 or 3), NR′—S(O)_{w}, and —S(O)_{w}—NR′R″ (where w is 1, 2 or 3)), C₃₋₆cycloalkyl, —S(O)_{w}—C₁₋₆alkyl (where w is 1, 2 or 3), —S(O)_{w}—NR′R″ (where w is 1, 2 or 3), and —NR′—S(O) (where w is 1, 2 or 3)),

5-6 membered heteroaryl having one, two, or three heteroatoms each independently selected from O, N and S (wherein the 5-6 membered heteroaryl may be optionally substituted on a carbon with one, two, three or more substituents selected from the group consisting of halogen, hydroxyl, nitro, cyano, carboxy, C₁₋₆alkyl, C₂₋₆alkenyl, C₂₋₆alkynyl, C₁₋₆alkoxy, phenyl (optionally substituted by one, two or three substituents each independently selected from the group consisting of halogen, hydroxyl, C₁₋₆alkyl, C₂₋₆alkenyl, C₂₋₆alkynyl, C₁₋₆alkoxy, NR′R″, C(O)—NR′R″, —C(O)—C₁₋₆alkyl, —C(O)—OH, —C(O)—C₁₋₆alkoxy, —S(O)_{w}—C₁₋₆alkyl (where w is 1, 2 or 3), —NR′—S(O)_{w}, and —S(O)_{w}—NR′R″ (where w is 1, 2 or 3)), heteroaryl, heterocycle, NR′R″, —C(O)—NR′R″, —C(O)—C₁₋₆alkyl, —C(O)—C₁₋₆alkoxy, —S(O)_{w}—C₁₋₆alkyl (where w is 1, 2 or 3), —NR′—S(O)_{w}, and —S(O)_{w}—NR′R″ (where w is 1, 2 or 3), and on a nitrogen by R′),

C₁₋₆alkyl, C₁₋₆alkoxy, C₂₋₆alkenyl, C₃₋₁₀cycloalkyl (optionally substituted with one, two, three or more substituents selected from the group consisting of halogen, hydroxyl, nitro, cyano, carboxy, NR′R″, —C(O)—NR′R″, ═CNR′, C₁₋₆alkyl, C₁₋₆alkoxy, —C(O)—C₁₋₆alkyl, and —C(O)—C₁₋₆alkoxy, and wherein the C₃₋₁₀cycloalkyl may optionally be a bridged cycloalkyl)), and a 4-6 membered heterocycloalkyl having one or two heteroatoms each independently selected from O, N and S (wherein the 4-6 membered heterocycloalkyl may be optionally substituted with one, two, three or more substituents selected from the group consisting of halogen, hydroxyl, nitro, cyano, carboxy, NR′R″, —C(O)—NR′R″, C₁₋₆alkyl, C₁₋₆alkoxy, —C(O)—C₁₋₆alkyl, and —C(O)—C₁₋₆alkoxy);

R′ is selected, independently for each occurrence, from H, methyl, ethyl, propyl, phenyl, and benzyl;

R″ is selected, independently for each occurrence, from H, methyl, ethyl, propyl, butyl, carboxybenzyl, —C(O)-methyl and —C(O)-ethyl, or R′ and R″ taken together may form a 4-6 membered heterocycle;

each of moieties R⁴, R⁵, R⁶, R⁷, R⁸, R⁹, R¹⁰, and R¹¹ are independently selected for each occurrence from the group consisting of hydrogen, C₁₋₆alkyl, C₂₋₆alkenyl, C₂₋₆alkynyl, halogen, hydroxyl, nitro, cyano, NR′R″, —C(O)—NR′R″, —S(O)_{w}—C₁₋₆alkyl (where w is 1, 2 or 3), —NR′—S(O)_{w}, and —S(O)_{w}—NR′R″ (where w is 0, 1 or 2), C₁₋₆alkoxy, —C(O)—OH, —C(O)—C₁₋₆alkyl, and —C(O)—C₁₋₆alkoxy;

wherein for each occurrence, C₁₋₆alkyl may be optionally substituted with one, two, three or more substituents selected from the group consisting of halogen, hydroxyl, nitro, cyano, carboxy, C₂₋₆alkenyl, C₂₋₆alkynyl, C₁₋₆alkoxy, phenyl, NR′R″, —C(O)—NR′R″, S(O)_{w}-methyl (where w is 1, 2 or 3), —NR′—S(O)_{w}, and S(O)_{w}—NR′R″ (where w is 0, 1 or 2); C₁₋₆alkoxy may be optionally substituted with one, two, three or more substituents selected from the group consisting of halogen, hydroxyl, nitro, cyano, carboxy, C₁₋₆alkyl, phenyl, NR′R″, —C(O)—NR′R″, S(O)_{w}—C₁₋₆alkyl (where w is 1, 2 or 3), —NR′—S(O)_{w}, and S(O)_{w}—NR′R″ (where w is 0, 1 or 2); and C₃₋₆cycloalkyl may be optionally substituted with one, two, three or more substituents selected from the group consisting of halogen, hydroxyl, nitro, cyano, carboxy, C₁₋₆alkyl, C₁₋₆alkoxy, —C(O)—C₁₋₆alkyl, —C(O)—C₁₋₆alkoxy, and NR′R″; and pharmaceutically acceptable salts thereof.

Also described herein are methods for identifying a compound useful for the treatment of infection by hepatitis B virus (HBV) and/or for clinically curing infection by HBV, comprising (a) measuring the ability of the compound to modulate core protein-mediated regulation of cccDNA and (b) identifying the compound as useful for treating or clinically curing a hepatitis B infection based on the ability of the compound to modulate core protein-mediated regulation of DNA (e.g., cccDNA). The compound can modulate core protein-mediated regulation of DNA (e.g., cccDNA) by (a) modulating the structure of core protein; (b) modulating the function of core protein; (c) modulating the binding of core protein to DNA (e.g., cccDNA); (d) depleting the amount of free core protein dimer available to bind to DNA (e.g., cccDNA); (e) altering nuclear import or export of core protein; (f) altering an interaction between DNA (e.g., cccDNA) and a chromatin component; (g) altering an interaction between core protein and a chromatin component; (h) altering the rate, quantity, quality or stability of RNA expressed from DNA (e.g., cccDNA); (i) altering the stability or maintenance of DNA (e.g., cccDNA); (j) modulating an innate immune response against HBV; (k) altering the encapsidation of pgRNA; (l) recycling HBV nucleic acid back to the nucleus to form DNA (e.g., cccDNA); and/or (m) production of host proteins whose genes are modulated by Cp including interferon stimulated genes.

The ability of the compound to modulate core protein-mediated regulation of DNA (e.g., cccDNA) can be measured by (a) detecting a change in an amount of or state of core protein bound to cccDNA, optionally by using a chromatin immunoprecipitation (ChIP) assay or immunoprecipitation and mass spectrometry; (b) performing a South-western blot of isolated DNA (e.g., cccDNA); (c) evaluating isolated DNA (e.g., cccDNA) by a qPCR endpoint or real-time reporter assay; (d) measuring viral antigen by ELISA; (e) measuring viral RNA by qRT-PCR; (f) performing an endpoint or real-time reporter assay; (g) performing an assay using energy transfer or quenching between labeled core protein and DNA (e.g., cccDNA) or between another cccDNA binding protein and DNA (e.g., cccDNA); (h) performing surface plasmon resonance (SPR); (i) performing biointerferometry; (j) performing a fluorescence-based method such as FRET, FP, or fluorescence quenching; and/or (k) production of host proteins whose genes are modulated by Cp, including interferon stimulated genes.

In some embodiments, the ability of the compound to affect core protein and thus modulate core protein-mediated regulation of DNA (e.g., cccDNA) also can be measured by assessing binding of the compound to a core protein dimer to determine whether the compound affects binding interactions of DNA (e.g., cccDNA) to the core protein dimer. In another embodiment, the ability of the compound to affect core protein and thus modulate core protein-mediated regulation of cccDNA can be measured using an assay comprising differentially reporter-tagged core protein subunits or by measuring in vitro binding of core protein to DNA (e.g., cccDNA), optionally comprising a competition assay with control DNA. In certain embodiments, the assay can comprise measuring the presence or quantity of a viral protein (e.g., HBsAg or HBeAg) or viral RNA. In some embodiments, the ability of the compound to affect core protein and thus modulate core protein-mediated regulation of DNA (e.g., cccDNA) is determined by differential scanning fluorimetry, isothermal calorimetry, thermopheresis, or Saturation Transfer Difference NMR. The method can also include varying the concentration of the compound until the compound modulates core protein-DNA (e.g., cccDNA) interaction.

Described herein are methods for identifying a compound useful for the treatment of infection by hepatitis B virus (HBV) and/or for clinically curing infection by HBV, comprising (a) measuring the ability of the one or more compounds to modulate core protein assembly; and (b) identifying the compound as useful for treating or clinically curing a hepatitis B infection based on the ability of the compound to modulate core protein. The method can also include measuring the ability of the compound to modulate core protein-mediated regulation of cccDNA.

In certain embodiments, the ability of the compound to modulate core protein is measured by assessing binding of a labeled compound to a core protein dimer to determine whether the compound affects binding interactions of DNA (e.g., cccDNA) to the core protein dimer. In other embodiments, the ability of the compound to modulate core protein is determined by measuring fluorescence quenching of labeled core protein. The ability of the compound to modulate core protein can be measured by measuring altered binding of core protein to antibodies or other proteins sensitive to Cp tertiary or quaternary structure, immunoprecipitation and Western blot, sandwich ELISA, and/or a BRET assay.

In certain embodiments, the DNA is cccDNA.

### BRIEF DESCRIPTION OF THE DRAWINGS

FIG. 1 is a schematic showing that Cp is involved in multiple aspects of the HBV life cycle.

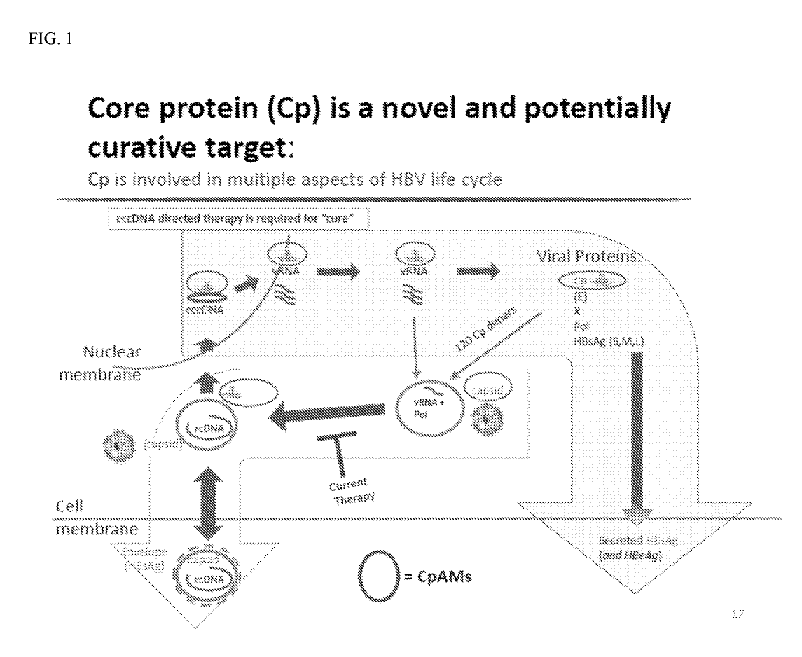

FIG. 2 shows that FL-HAP binds capsid with high affinity. FL-HAP was added to core protein dimer, which assembled. The mixture was resolved on a Superose 6 column. Absorbance monitored at 495 nm shows FL-HAP binding to and co-migrating with the capsid and large oligomer.

FIG. 3A-C shows a fluorescence quenching assay for depletion of free dimer. (A) Dimers have cysteines engineered at the C-termini, at either end, which is labeled with BoDIPY-FL. (B) Dimers are fluorescent (left) capsids (right) are not. (C) The change in fluorescence matches other methods of monitoring assembly, such as light scattering (LS), as shown.

FIG. 4 shows elution of Cp149 from a Superose 6 column, demonstrating how a CpAM-emulating Cp mutant can alter Stokes' radius.

### DETAILED DESCRIPTION

### Methods of Treatment

Described herein are methods for treating or clinically curing a patient infected by hepatitis B virus (HBV), including administering to the patient a therapeutically effective amount of a compound capable of modulating core protein-mediated regulation of DNA (e.g., cccDNA) in an HBV infected cell of the patient and/or modulating core protein assembly. In some embodiments, administration of the two compounds results in an improved clinical outcome compared to administration of either compound alone. In other embodiments, administration of the two compounds results in a synergistically improved clinical outcome compared to administration of either compound alone.

Also described herein are methods for treating or clinically curing a patient infected by hepatitis B virus (HBV), the method comprising the step of administering to the patient a compound capable of modulating core protein assembly, wherein the compound is administered at a dose sufficient to modulate core protein-mediated regulation of DNA (e.g., cccDNA). In some embodiments, administration of the compound alters HBV's S antigen (HBsAg), E antigen (HBeAg), or viral RNA levels. Methods for measuring HBsAg, HBeAg and RNA are well known in the art. For example methods for measuring HBsAg are described in Ly et al. (2006) J Clin Microbiol. 44(7): 2321-2326. Methods for measuring HBV DNA are found at www.accessdata.fda.gov/cdrh_docs/pdf8/P080026a.pdf. Methods for measuring HBeAg (and HBsAg) are described in Lee et al. (2011) Hepatology 53(5):1486-93. Methods for measuring viral RNA, e.g., pgRNA, are described in Lu et al. (2009) J Viral Hepat. 16(2):104-12; and Bai et al. (2013) Int J Hepatol. 2013:849290.

In certain embodiments the compound is capable of (a) modulating the structure of core protein; (b) modulating the function of core protein; (c) modulating the binding of core protein to DNA (e.g., cccDNA); (d) depleting the amount of free core protein dimer available to bind to cccDNA; (e) altering nuclear import or export of core protein; (f) altering an interaction between DNA (e.g., cccDNA) and a chromatin component; (g) altering an interaction between core protein and a chromatin component; (h) altering the rate, quantity, quality or stability of RNA expressed from DNA (e.g., cccDNA); (i) altering the stability or maintenance of DNA (e.g., cccDNA); and/or (j) modulating an innate immune response against HBV. In certain embodiments, the compound acts allosterically or orthosterically.

In some embodiments, the method also includes administering an additional compound. For example, one or more of the following compounds can be administered in combination with the compounds described herein: a nucleoside or nucleotide HBV polymerase inhibitor, a modified nucleic acid, a peptide entry inhibitor, an interferon (Type I, II or III), a lymphotoxin beta agonist, a Toll-like receptor agonist, a non-nucleoside small molecule HBV polymerase inhibitor, a non-nucleotide small molecule HBV polymerase inhibitor, a compound affecting capsid maturation, an HBV DNA (e.g., cccDNA) transcriptional modulator, a DNA (e.g., cccDNA) biosynthesis inhibitor, a subviral particle secretion inhibitor, a checkpoint modulator, an siRNA, a therapeutic vaccine, an entry inhibitor, a transcriptional modifier, a topoisomerase inhibitor, a compound that modulates presentation of HBV antigen via MHC, an HBV RNase H inhibitor, a proteasome inhibitor, a cyclophilin inhibitor, a transcription activator-like effector nuclease (TALEN), a DNA cleavage enzyme targeting HBV DNA (e.g., cccDNA), a dominant negative HBV mutant, a second mitochondrial-derived activator of caspases (SMAC) mimetic, nucleic acid-based polymer (NAP) such as REP-2139Ca and REP-2055, a Stimulator of Interferon Genes (STING) such as DMXAA and 2′3′-cGAMP, and an inhibitory peptide.

Nucleoside or nucleotide HBV polymerase inhibitors include, e.g., lamivudine (3TC, LMV), telbivudine, adefovir (ADV), entecavir (ETV), tenofovir, LB80380, lagociclovir valactate, pradefovir, emtricitabine, valtorcitabine, and amdoxovir. See, e.g., Hu et al. (2013) Annual Reports in Medicinal Chemistry 48:265-281. Modified nucleic acids include REP 9AC′. Id. Entry inhibitors include peptide entry inhibitors, e.g., Myrcludex-B. Id. See also, Interferons include type I, II or III interferons, e.g., Type III interferons (Interferon lambda). Id. Exemplary lymphotoxin beta agonists include those discussed in Lucifora et al. (2014) Science 343(6176):1221-8. Toll-like receptor agonists include, e.g., the Toll-like receptor-7 (TLR-7) agonist GS-9620. See, e.g., Hu et al. (2013) Annual Reports in Medicinal Chemistry 48:265-281. Non-nucleoside/nucleotide small molecule HBV polymerase inhibitors include foscarnet, oxymatrine, and compounds 10 and 11

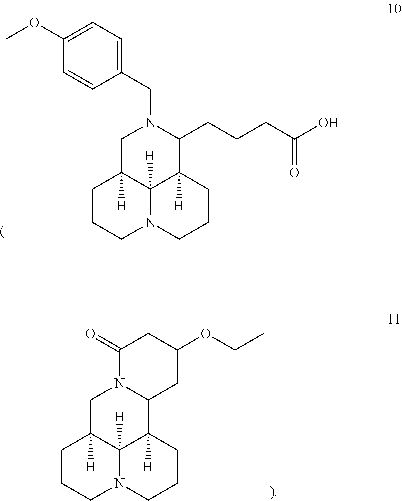

Id. Compounds affecting capsid maturation include, e.g., GLS-4 and HAP12. HBV cccDNA transcriptional modulators include, e.g., helioxanthin and derivatives 14 and 15

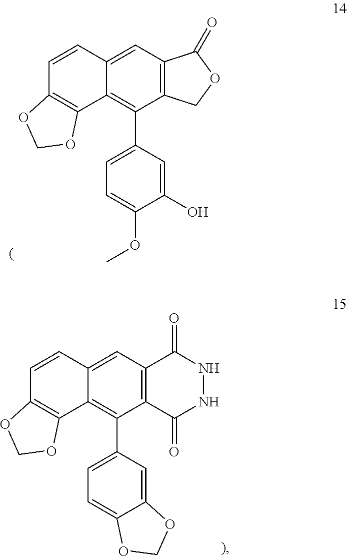 [SMILES: COc1ccc(-c2c3c(cc4ccc5c(c24)OCO5)C(=O)OC3)cc1O ; O=c1[nH][nH]c(=O)c2c(-c3ccc4c(c3)OCO4)c3c4c(ccc3cc12)OCO4]

quinolin-2-one and analog 17

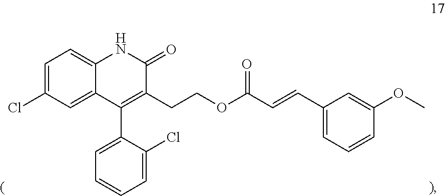 [SMILES: COc1cccc(/C=C/C(=O)OCCc2c(-c3ccccc3Cl)c3cc(Cl)ccc3[nH]c2=O)c1]

caudatin, and related compound 19

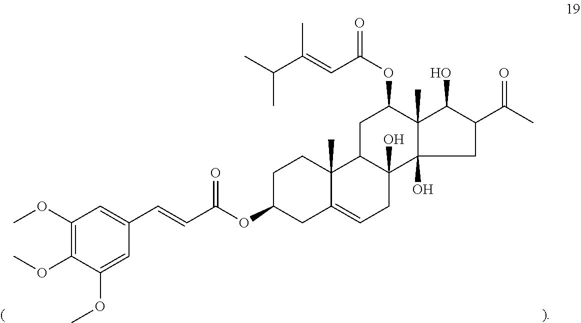

See, e.g., Hu et al. (2013) Annual Reports in Medicinal Chemistry 48:265-281. cccDNA biosynthesis inhibitors include, e.g., sulfonamides CCC-0975 and CCC-0346. Id. Agents that block pgRNA encapsidation include isothiafludine (NZ-4), naphthylureas of the carbonyl J acid family (e.g., KM-1), and cIAP2. Subviral particle secretion inhibitors include HBF-0529, BM601 benzimidazole, and PBHBV-2-15. Id. Checkpoint modulators include an anti-PD-1 antibody, an anti-PD-L1 antibody, or an anti-CTLA4 antibody. siRNAs include siRNAs against HBV (e.g., ISIS-HBV mRNA-targeted antisense) or host RNA. See, also, e.g., Wooddell et al. (2013) Mol. Ther. 21(5):973-85. Therapeutic vaccines include GS-4774 (GlobeImmune) and those described in Obeng-Adjei et al. (2013) Cancer Gene Ther. 20(12):652-62. Transcriptional modifiers include Tenofovir and Entecavir. (See, e.g., Kitrinos et al. (2014) Hepatology 59(2):434-42. Topoisomerase inhibitors include, e.g., Topo II inhibitors, such as etoposide, and Topo I inhibitors, such as Irinitecan. Compounds that modulate presentation of HBV antigen via MHC include, e.g., chloroquine. (See, e.g., Accapezzato et al. (2005) J Exp Med. 202(6):817-28.) Methods for designing HBV RNase H inhibitors are discussed in Hayer et al. (2014) J Virol. 88(1):574-82. Proteasome inhibitors include, e.g., MG132. See, e.g., Wang et al. (2013) Hepatogastroenterology. 60(124):837-41. Cyclophilin inhibitors include, e.g., NVP-018 (Neurovive® Pharmaceutical AB), SCY-635, alisporivir, Sanglifehrin A derivatives, and OCB-030. Transcription activator-like effector nucleases (TALENs) targeting the HBV genome are described in, e.g., Bloom et al. (2013) Mol Ther. 21(10):1889-1897. DNA cleavage enzymes targeting HBV cccDNA are described in, e.g., Schiffer et al. (2013) DOI: 10.1371/journal.pcbi.1003131. Dominant negative HBV mutants are described in the art. Second mitochondrial-derived activator of caspases (SMAC) mimetics include birinapant (TL32711), LCL161 (Novartis), GDC-0917 (Genentech), HGS1029 (Human Genome Sciences), and AT-406 (Ascenta). Nucleic acid-based polymers (NAPs) include REP-2139Ca and REP-2055. Stimulator of Interferon Genes (STINGs) include DMXAA and 2′3′-cGAMP. Inhibitory peptides (e.g., NH₂—SFYSVLFLWG TCGGFSHSWY-COOH; NH₂-LCETVRFWPV CFCSLYVICS-COOH; NH₂—SCAPAWSPAP TVVFVALYVV-COOH; NH₂-QWGMDSLIRL YLWESLGLLS-COOH; NH₂-IHPLSRGNFF PHVRLMGEWR-COOH; NH₂-GQALCAGVSL FADWLHESTL-COOH; NH₂-LKHFDPRWPL MSLMSSWACM-COOH; NH₂-PPLRKAFCWR CFNWLSTKRL-COOH; and NH₂-LRKSMLKVGR DVCYVSLWVF-COOH) are described in International Patent Publication WO2000/042063.

### Compounds

The methods described herein can include administration of any one of the following compounds:

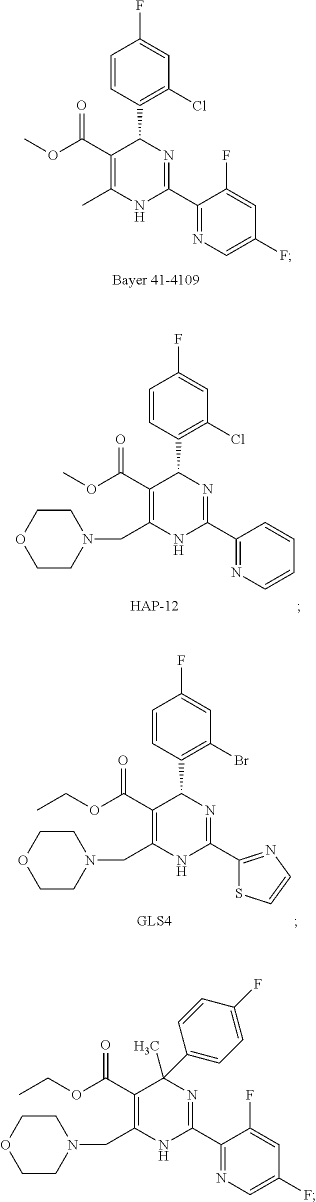 [SMILES: COC(=O)C1=C(C)NC(c2ncc(F)cc2F)=N[C@H]1c1ccc(F)cc1Cl ; COC(=O)C1=C(CN2CCOCC2)NC(c2ccccn2)=N[C@H]1c1ccc(F)cc1Cl ; "HAP-12" ; CCOC(=O)C1=C(CN2CCOCC2)NC(c2nccs2)=N[C@H]1c1ccc(F)cc1Br ; "GLS4" ; CCOC(=O)C1=C(CN2CCOCC2)NC(c2ncc(F)cc2F)=NC1(C)c1ccc(F)cc1]

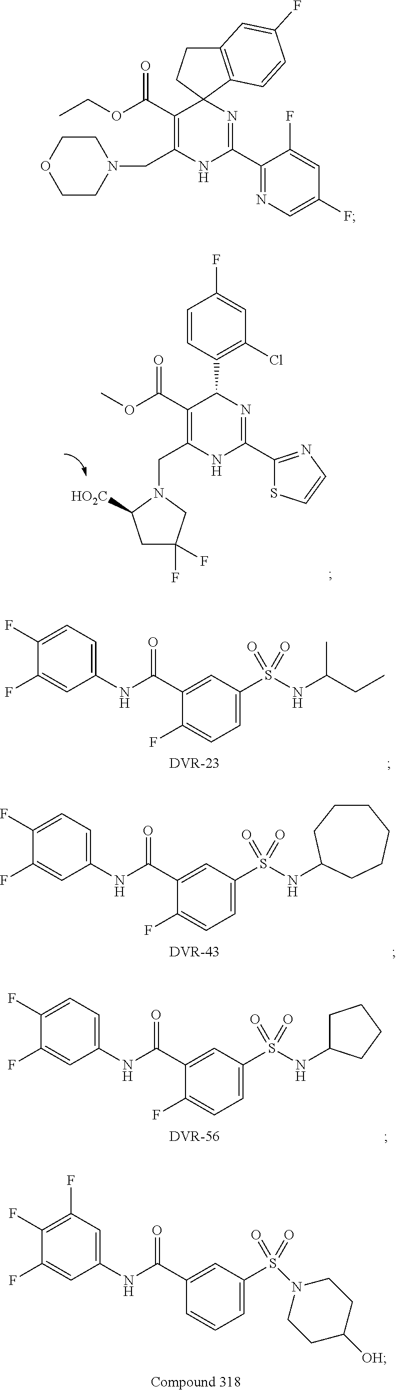

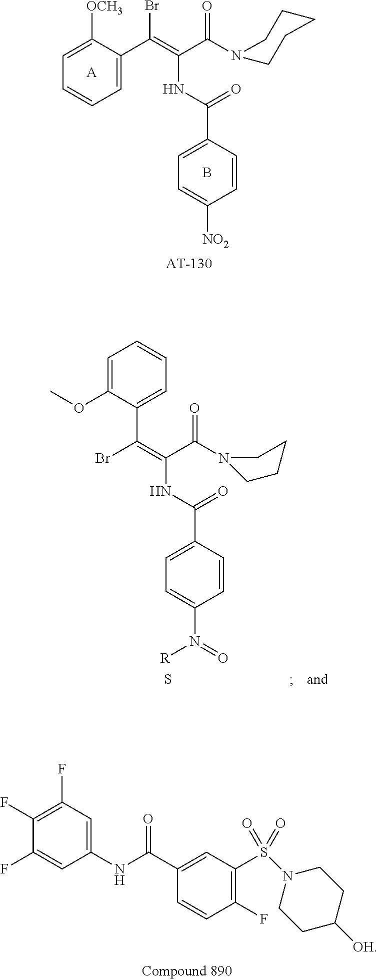

The methods described herein can include administration of any of the capsid promoting and HBsAg reducing molecules described in International Publication No. WO/2015/057945, U.S. Provisional Patent Application No. 62/148,994, and in International Patent Application No. PCT/US2015/020444.

For example, the methods described herein can include administration of a compound of Formula 1 (from International Publication No. WO/2015/057945) having the structure:

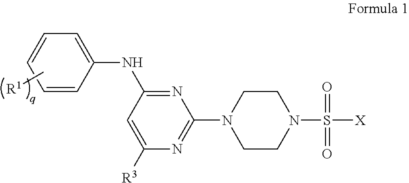

or a pharmaceutically acceptable salt or stereoisomer thereof, wherein:
X is selected from the group consisting of

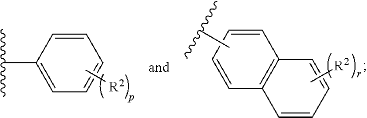

q is 0, 1, 2, 3 or 4;
p is 0, 1, 2, 3, or 4;
r is 0, 1, 2, 3, or 4;
R¹ is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″);
w is 0, 1 or 2;
R′ is independently for each occurrence selected from the group consisting of —H and —C₁-C₆alkyl;
R″ is independently for each occurrence selected from the group consisting of —H and —C₁-C₆alkyl; or R′ and R″ are taken together with the nitrogen atom to which they are attached to form a 4-7 membered heterocyclic ring;
R² is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″); and
R³ is selected from the group consisting of —H and —C₁-C₆alkyl;

wherein C₁-C₆alkyl or C₁-C₆alkoxy may be independently for each occurrence optionally substituted with one, two, or three halogens.

The methods described herein can also include a compound of Formula 1 (described in U.S. Provisional Patent Application No. 62/148,994) having the structure:

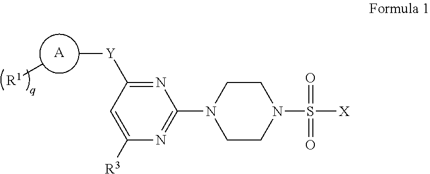

or a pharmaceutically acceptable salt or stereoisomer thereof, wherein:

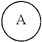

is selected from the group consisting of phenyl, naphthyl, and heteroaryl;
Y is selected from the group consisting of a bond, —O—, —S(O)_{w}—, and —N(R′)—;
X is selected from the group consisting of phenyl, naphthyl, and heteroaryl; wherein X
is optionally substituted with one, two, three, or four R² groups;
provided that at least one of

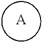

or X is a heteroaryl;
R¹ is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″);
q is 0, 1, 2, 3 or 4;
w is 0, 1 or 2;
R′ is independently for each occurrence selected from the group consisting of —H and —C₁-C₆alkyl;
R″ is independently for each occurrence selected from the group consisting of —H and —C₁-C₆alkyl; or R′ and R″ are taken together with the nitrogen atom to which they are attached to form a 4-7 membered heterocyclic or heteroaryl ring, each of which is optionally substituted with an oxo group;
R² is independently for each occurrence selected from the group consisting of —H, —C₁-C₆alkyl, —C₁-C₆alkoxy, —C₁-C₆alkyl-O—C₁-C₆alkyl, halogen, oxo, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″); and
R³ is selected from the group consisting of —H, —C₁-C₆alkyl, —N(R′)(R″), —N(R′)C₁-C₆alkyl-N(R′)(R″), —N(R′)—C₁-C₆alkyl-OR′, —OH, —C₁-C₆alkoxy, —O—C₁-C₆alkyl-OR′, —O-heterocyclyl, —O-heteroaryl, —O—C₁-C₆alkyl-heteroaryl, —C₁-C₆alkyl-heteroaryl, heterocyclyl, and heteroaryl, wherein heterocyclyl and heteroaryl are optionally substituted with one or two C₁-C₆alkyl or halogen;
wherein C₁-C₆alkyl or C₁-C₆alkoxy may be independently for each occurrence optionally substituted with one, two, or three halogens.

The methods described herein can also include a compound of Formula 2 (described in U.S. Provisional Patent Application No. 62/148,994) having the structure:

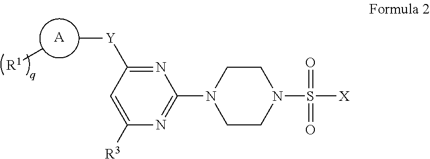

or a pharmaceutically acceptable salt or stereoisomer thereof, wherein:

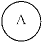

is selected from the group consisting of phenyl, naphthyl, and heteroaryl;
Y is selected from the group consisting of a bond, —O—, and —S(O)_{w}—;
X is selected from the group consisting of phenyl, naphthyl, and heteroaryl; wherein X is optionally substituted with one, two, three, or four R² groups;
R¹ is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″);
q is 0, 1, 2, 3 or 4;
w is 0, 1 or 2;
R′ is independently for each occurrence selected from the group consisting of —H and —C₁-C₆alkyl;
R″ is independently for each occurrence selected from the group consisting of —H and —C₁-C₆alkyl; or R′ and R″ are taken together with the nitrogen atom to which they are attached to form a 4-7 membered heterocyclic or heteroaryl ring, each of which is optionally substituted with an oxo group;
R² is independently for each occurrence selected from the group consisting of —H, —C₁-C₆alkyl, —C₁-C₆alkoxy, —C₁-C₆alkyl-O—C₁-C₆alkyl, halogen, oxo, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″); and
R³ is selected from the group consisting of —H, —C₁-C₆alkyl, —N(R′)(R″), —N(R′)C₁-C₆alkyl-N(R′)(R″), —N(R′)—C₁-C₆alkyl-OR′, —OH, —C₁-C₆alkoxy, —O—C₁-C₆alkyl-OR′, —O-heterocyclyl, —O-heteroaryl, —O—C₁-C₆alkyl-heteroaryl, —C₁-C₆alkyl-heteroaryl, heterocyclyl, and heteroaryl, wherein heterocyclyl and heteroaryl are optionally substituted with one or two C₁-C₆alkyl or halogen;
wherein C₁-C₆alkyl or C₁-C₆alkoxy may be independently for each occurrence optionally substituted with one, two, or three halogens.

The methods described herein can also include a compound of Formula 3 (described in U.S. Provisional Patent Application No. 62/148,994) having the structure:

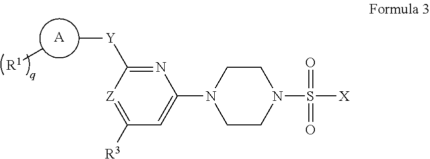

or a pharmaceutically acceptable salt or stereoisomer thereof, wherein:

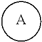

is selected from the group consisting of phenyl, naphthyl, and heteroaryl;
Y is selected from the group consisting of a bond, —O—, —S(O)_{w}—, and —N(R′)—;
Z is CH or N;
X is selected from the group consisting of phenyl, naphthyl, and heteroaryl; wherein X is optionally substituted with one, two, three, or four R² groups;
R¹ is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″);
q is 0, 1, 2, 3 or 4;
w is 0, 1 or 2;
R′ is independently for each occurrence selected from the group consisting of —H and —C₁-C₆alkyl;
R″ is independently for each occurrence selected from the group consisting of —H and —C₁-C₆alkyl; or R′ and R″ are taken together with the nitrogen atom to which they are attached to form a 4-7 membered heterocyclic or heteroaryl ring, each of which is optionally substituted with an oxo group;
R² is independently for each occurrence selected from the group consisting of —H, —C₁-C₆alkyl, —C₁-C₆alkoxy, —C₁-C₆alkyl-O—C₁-C₆alkyl, halogen, oxo, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″); and
R³ is selected from the group consisting of —H, —C₁-C₆alkyl, —N(R′)(R″), —N(R′)C₁-C₆alkyl-N(R′)(R″), —N(R′)—C₁-C₆alkyl-OR′, —OH, —C₁-C₆alkoxy, —O—C₁-C₆alkyl-OR′, —O-heterocyclyl, —O-heteroaryl, —O—C₁-C₆alkyl-heteroaryl, —C₁-C₆alkyl-heteroaryl, heterocyclyl, and heteroaryl, wherein heterocyclyl and heteroaryl are optionally substituted with one or two C₁-C₆alkyl or halogen;
wherein C₁-C₆alkyl or C₁-C₆alkoxy may be independently for each occurrence optionally substituted with one, two, or three halogens.

The methods described herein can also include a compound of Formula 4 (described in U.S. Provisional Patent Application No. 62/148,994) having the structure:

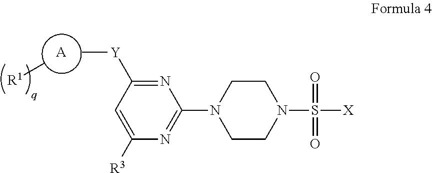

or a pharmaceutically acceptable salt or stereoisomer thereof, wherein:

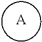

is selected from the group consisting of phenyl, naphthyl, and heteroaryl;
Y is selected from the group consisting of a bond, —O—, —S(O)_{w}—, and —N(R′)—;
X is selected from the group consisting of phenyl, naphthyl, and heteroaryl; wherein X is optionally substituted with one, two, three, or four R² groups;
R¹ is independently for each occurrence selected from the group consisting of —H, —C₁-C₆alkyl, —C₁-C₆alkoxy, —C₁-C₆alkyl-O—C₁-C₆alkyl, halogen, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″);
q is 0, 1, 2, 3 or 4;
w is 0, 1 or 2;
R′ is independently for each occurrence selected from the group consisting of —H and —C₁-C₆alkyl;
R″ is independently for each occurrence selected from the group consisting of —H and —C₁-C₆alkyl; or R′ and R″ are taken together with the nitrogen atom to which they are attached to form a 4-7 membered heterocyclic or heteroaryl ring, each of which is optionally substituted with an oxo group;
R² is independently for each occurrence selected from the group consisting of —H, —C₁-C₆alkyl, —C₁-C₆alkoxy, —C₁-C₆alkyl-O—C₁-C₆alkyl, halogen, oxo, cyano, —OH, —C(O)H, —CO₂R′, —C(O)N(R′)(R″), —C(O)C₁-C₆alkyl, —N(R′)(R″), —NO₂, —N(R)C(O)C₁-C₆alkyl, —S(O)_{w}—C₁-C₆alkyl, —N(R)S(O)_{w}—C₁-C₆alkyl, and —S(O)_{w}—N(R′)(R″); and
R³ is selected from the group consisting of —N(R′)(R″), —N(R)C₁-C₆alkyl-N(R′)(R″), —N(R)—C₁-C₆alkyl-OR′, —OH, —C₁-C₆alkoxy, —O—C₁-C₆alkyl-OR′, —O-heterocyclyl, —O-heteroaryl, —O—C₁-C₆alkyl-heteroaryl, —C₁-C₆alkyl-heteroaryl, heterocyclyl, and heteroaryl, wherein heterocyclyl and heteroaryl are optionally substituted with one or two C₁-C₆alkyl or halogen;
wherein C₁-C₆alkyl or C₁-C₆alkoxy may be independently for each occurrence optionally substituted with one, two, or three halogens

The methods described herein can also include a compound of Formula 4 (described in International Patent Application No. PCT/US2015/020444) having the structure:

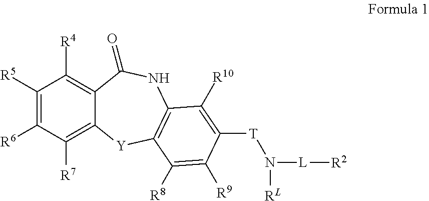

wherein:

T is selected from the group consisting of —C(O)—, —CH₂—C(O)—, —N(C(O)—CH₃)—, —NH—, —O—, and S(O)_{z}—, where z is 0, 1, or 2;

Y is C(R¹¹)₂, S(O)_{y}, NR_{Y} and O wherein y is 0, 1, or 2;

R_{Y} is selected from the group consisting of H, methyl, ethyl, propyl, phenyl and benzyl;

R_{L} is selected from the group consisting of H, methyl, and —C(O)—C₁₋₃alkyl;

L is a bond or C₁₋₄ straight chain alkylene optionally substituted by one or two substituents each independently selected from the group consisting of methyl (optionally substituted by halogen or hydroxyl), ethenyl, hydroxyl, NR′R″, phenyl, heterocycle, and halogen and wherein the C₁₋₄ straight chain alkylene may be interrupted by an —O—;

R² is selected from the group consisting of H, phenyl or naphthyl (wherein the phenyl or naphthyl may be optionally substituted with one, two, three or more substituents selected from the group consisting of halogen, hydroxyl, nitro, cyano, carboxy, C₁₋₆alkyl, C₂₋₆alkenyl, C₂₋₆alkynyl, C₁₋₆alkoxy, NR′R″, —C(O)—NR′R″, —C(O)—C₁₋₆alkyl, —C(O)—C₁₋₆alkoxy, phenyl (optionally substituted by one, two or three substituents each independently selected from the group consisting of halogen, hydroxyl, cyano, C₁₋₆alkyl, C₂₋₆alkenyl, C₂₋₆alkynyl, C₁₋₆alkoxy, NR′R″, —C(O)—NR′R″, —C(O)—C₁₋₆alkyl, —C(O)—C₁₋₆alkoxy, —S(O)_{w}—C₁₋₆alkyl (where w is 1, 2, or 3), S(O)_{w}—NR′R″ (where w is 1, 2 or 3), —NR′—S(O)_{w}, and —S(O)_{w}—NR′R″ (where w is 1, 2, or 3)), heteroaryl (optionally substituted by one, two or three substituents each independently selected from the group consisting of halogen, hydroxyl, C₁₋₆alkyl, C₂₋₆alkenyl, C₂₋₆alkynyl, C₁₋₆alkoxy, NR′R″, —C(O)—NR′R″, —C(O)—C₁₋₆alkyl, —C(O)—C₁₋₆alkoxy, —S(O)_{w}—C₁₋₆alkyl (where w is 1, 2, or 3), NR′—S(O)_{w}, and —S(O)_{w}—NR′R″ (where w is 1, 2, or 3)), C₃₋₆cycloalkyl, —S(O)_{w}—C₁₋₆alkyl (where w is 1, 2, or 3), —S(O)_{w}—NR′R″ (where w is 1, 2, or 3), and —NR′—S(O) (where w is 1, 2, or 3)), 5-6 membered heteroaryl having one, two, or three heteroatoms each independently selected from O, N, and S (wherein the 5-6 membered heteroaryl may be optionally substituted on a carbon with one, two, three or more substituents selected from the group consisting of halogen, hydroxyl, nitro, cyano, carboxy, C₁₋₆alkyl, C₂₋₆alkenyl, C₂₋₆alkynyl, C₁₋₆alkoxy, phenyl (optionally substituted by one, two or three substituents each independently selected from the group consisting of halogen, hydroxyl, C₁₋₆alkyl, C₂₋₆alkenyl, C₂₋₆alkynyl, C₁₋₆alkoxy, NR′R″, —C(O)—NR′R″, —C(O)—C₁₋₆alkyl, —C(O)—OH, —C(O)—C₁₋₆alkoxy, —S(O)_{w}—C₁₋₆alkyl (where w is 1, 2, or 3), —NR′—S(O)_{w}, —S(O)_{w}—NR′R″ (where w is 1, 2, or 3)), heteroaryl, heterocycle, NR′R″, —C(O)—NR′R″, —C(O)—C₁₋₆alkyl, —C(O)—C₁₋₆alkoxy, —S(O)_{w}—C₁₋₆alkyl (where w is 1, 2, or 3), —NR′—S(O)_{w}, and —S(O)_{w}—NR′R″ (where w is 1, 2, or 3), and on a nitrogen by R′), C₁₋₆alkyl, C₁₋₆alkoxy, C₂₋₆alkenyl, C₃₋₁₀cycloalkyl (optionally substituted with one, two, three, or more substituents selected from the group consisting of halogen, hydroxyl, nitro, cyano, carboxy, NR′R″, —C(O)—NR′R″, ═CNR′, C₁₋₆alkyl, C₁₋₆alkoxy, —C(O)—C₁₋₆alkyl, and —C(O)—C₁₋₆alkoxy, and wherein the C₃₋₁₀cycloalkyl may optionally be a bridged cycloalkyl)), and a 4-6 membered heterocycloalkyl having one or two heteroatoms each independently selected from O, N, and S (wherein the 4-6 membered heterocycloalkyl may be optionally substituted with one, two, three, or more substituents selected from the group consisting of halogen, hydroxyl, nitro, cyano, carboxy, NR′R″, —C(O)—NR′R″, C₁₋₆alkyl, C₁₋₆alkoxy, —C(O)—C₁₋₆alkyl, and —C(O)—C₁₋₆alkoxy);

R′ is selected, independently for each occurrence, from H, methyl, ethyl, propyl, phenyl, and benzyl;

R″ is selected, independently for each occurrence, from H, methyl, ethyl, propyl, butyl, carboxybenzyl, —C(O)-methyl and —C(O)-ethyl, or R′ and R″ taken together may form a 4-6 membered heterocycle;

each of moieties R⁴, R⁵, R⁶, R⁷, R⁸, R⁹, R¹⁰, and R¹¹ are independently selected for each occurrence from the group consisting of hydrogen, C₁₋₆alkyl, C₂₋₆alkenyl, C₂₋₆alkynyl, halogen, hydroxyl, nitro, cyano, NR′R″, —C(O)—NR′R″, —S(O)_{w}—C₁₋₆alkyl (where w is 1, 2, or 3), —NR′—S(O)_{w}, and —S(O)_{w}—NR′R″ (where w is 0, 1, or 2), C₁₋₆alkoxy, —C(O)—OH, —C(O)—C₁₋₆alkyl, and —C(O)—C₁₋₆alkoxy;

wherein for each occurrence, C₁₋₆alkyl may be optionally substituted with one, two, three, or more substituents selected from the group consisting of halogen, hydroxyl, nitro, cyano, carboxy, C₂₋₆alkenyl, C₂₋₆alkynyl, C₁₋₆alkoxy, phenyl, NR′R″, —C(O)—NR′R″, S(O)_{w}-methyl (where w is 1, 2, or 3), —NR′—S(O)_{w}, and S(O)_{w}—NR′R″ (where w is 0, 1, or 2); C₁₋₆alkoxy may be optionally substituted with one, two, three, or more substituents selected from the group consisting of halogen, hydroxyl, nitro, cyano, carboxy, C₁₋₆alkyl, phenyl, NR′R″, —C(O)—NR′R″, S(O)_{w}—C₁₋₆alkyl (where w is 1, 2 or 3), —NR′—S(O)_{w}, and S(O)_{w}—NR′R″ (where w is 0, 1, or 2); and C₃₋₆cycloalkyl may be optionally substituted with one, two, three, or more substituents selected from the group consisting of halogen, hydroxyl, nitro, cyano, carboxy, C₁₋₆alkyl, C₁₋₆alkoxy, —C(O)—C₁₋₆alkyl, —C(O)—C₁₋₆alkoxy, and NR′R″; and pharmaceutically acceptable salts thereof.

In one illustrative embodiment of the disclosure, compounds described in International Patent Publication WO2001/068641 can be used, including a compound having the formula:

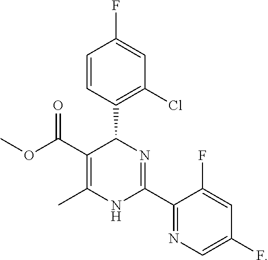 [SMILES: COC(=O)C1=C(C)NC(c2ncc(F)cc2F)=N[C@H]1c1ccc(F)cc1Cl]

In another illustrative embodiment of the disclosure, compounds described in International Patent Publication WO2013/019967, including a compound having the formula

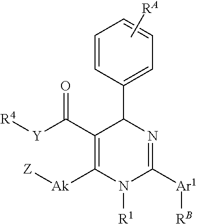

or a pharmaceutically acceptable salt thereof is described, wherein
Ar¹ is selected from the group consisting of phenyl, 2-pyridyl, 3-pyridyl, and 4-pyridyl;
R¹ is hydrogen or pro-drug forming group;
Ak is alkylene;

### Z is

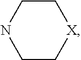

where X is CHN₃, C═O, C═NR⁵, —C(O)N(R^{N})—, or NR^{N}, where R⁵ is hydroxy or a derivative thereof or amino or a derivative thereof, and R^{N} is selected from the group consisting of alkyl, alkenyl, alkynyl, heteroalkyl, arylalkyl, heteroarylalkyl, alkyl-C(O), heteroalkyl-C(O), alkoxyl-C(O), alkynyl-C(O), alkylacylamino-C(O), and heteroalkylacylamino-C(O), each of which is optionally substituted;
R⁴ is alkyl, heteroalkyl, alkenyl, or alkynyl, each of which is optionally substituted;

### Y is O or HN;

R^{A} represents from 0 to 3 substituents independently in each instance, halo or selected from the group consisting of alkyl, heteroalkyl, aryl, heteroaryl, amino and derivatives thereof, and hydroxyl and derivatives thereof, each of which is optionally substituted; and
R^{B} represents from 0 to 3 substituents independently in each instance, halogen or selected from the group consisting of alkyl, heteroalkyl, aryl, heteroaryl, amino and derivatives thereof, and hydroxyl and derivatives thereof, each of which is optionally substituted.

In another illustrative embodiment of the disclosure, compounds described in Bourne et al. (2008) J. Vir. 82(20):10262-10270 and in International Patent Publication WO2013/019967 can be used in accordance with the methods of the disclosure, including a compound having the formula

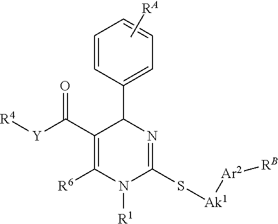

or a pharmaceutically acceptable salt thereof is described, wherein;
Ar² is aryl or heteroaryl each of which is optionally substituted;
R¹ is independently in each instance selected from the group consisting of hydrogen and pro-drug forming group;
R⁴ is alkyl, heteroalkyl, alkenyl, or alkynyl, each of which is optionally substituted;

### Y is O or HN;

R⁶ is in each instance independently selected from the group consisting of hydrogen and Ak-Z, where Ak is alkylene, and Z is independently in each instance hydrogen or NR₂R₃; where R₂ and R₃ are independently in each instance hydrogen, or selected from the group consisting of alkyl, cycloalkyl, heteroalkyl and heterocycloalkyl, each of which is optionally substituted, or R₂ and R₃ are taken together with the attached nitrogen to form

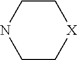

wherein X is CHN₃, C═O, —C(O)N(R^{Na})—, C═NR⁵, or NR^{Na} where R⁵ is hydroxy or a derivative thereof or amino or a derivative thereof; and RN^{a} is selected from the group consisting of hydrogen, and alkyl, alkenyl, alkynyl, heteroalkyl, arylalkyl, heteroarylalkyl, alkyl-C(O), heteroalkyl-C(O), alkoxyl-C(O), alkynyl-C(O), alkylacylamino-C(O), and heteroalkylacylamino-C(O), each of which is optionally substituted;
Ak¹ is (CH₂)n, where n is 1 to 4;
R^{A} represents from 0 to 3 substituents independently in each instance, halo or selected from the group consisting of alkyl, heteroalkyl, aryl, heteroaryl, amino and derivatives thereof, and hydroxyl and derivatives thereof, each of which is optionally substituted; and
R^{B} represents from 0 to 3 substituents independently in each instance, halogen or selected from the group consisting of alkyl, heteroalkyl, aryl, heteroaryl, amino and derivatives thereof, and hydroxyl and derivatives thereof, each of which is optionally substituted.

In another illustrative embodiment, compounds described in International Patent Publication WO2010/069147 can be used in accordance with the methods of the disclosure.

In another illustrative embodiment, compounds described in International Patent Publication WO2013/102655 can be used in accordance with the methods of the disclosure. For example, the present disclosure provides compounds, which can be represented by the formula I:

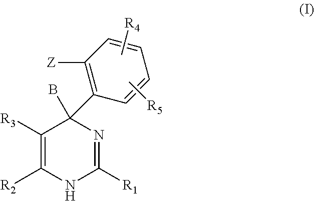

including any possible stereoisomers or tautomeric forms thereof, wherein:
B is selected from the group comprising C₁-C₃alkyl optionally substituted with one or more fluoro atoms;
Z is selected from H or halogen; or B and Z together with the carbons to which they are attached form a 4-7 membered ring, optionally containing one or more heteroatoms, wherein the 4-7 membered ring optionally is substituted with one or more substituents selected from the group comprising C₁-C₃alkyl, oxo, OH and halogen;
R₁ is selected from the group comprising heteroaryl and phenyl, optionally substituted with one or more substituents selected from the group comprising halogen and C₁-C₃alkyl;
R₂ is selected from the group comprising —R₆-R₇, C≡N, cyclopropyl, and CF₃;
R₃ is selected from the group comprising C₁-C₃alkoxycarbonyl, and C≡N;
R₄ and R₅ independently are selected from the group comprising H, methyl and halogen;
R₆ is C₁-C₃alkyl, C₂-C₃alkenyl, both optionally substituted with one or more fluoro;
R₇ is selected from the group comprising hydrogen, a hetero C₃₋₇cycloalkyl, cyclopropyl, C₁-C₃alkoxy and CF₃;
or a pharmaceutically acceptable salt or a solvate thereof.
B for example may be selected from the group comprising C₁-C₃alkyl optionally substituted with one or more fluoro atoms; Z is selected from H or halogen; or B and Z together with the carbons to which they are attached form a 4-7 membered ring, optionally containing one or more heteroatoms, wherein the 4-7 membered ring optionally is substituted with one or more substituents selected from the group comprising C₁-C₃alkyl, oxo and halogen;
R₁ is selected from the group comprising heteroaryl and phenyl, optionally substituted with one or more substituents selected from the group comprising halogen and C₁-C₃alkyl;
R₂ is selected from the group comprising —R₆-R₇, C≡N, cyclopropyl, and CF₃;
R₃ is selected from the group comprising C₁-C₃alkoxycarbonyl, and C≡N;
R₄ and R₅ independently are selected from the group comprising H and halogen;
R₆ is C₁-C₃alkyl, optionally substituted with fluoro;
and R₇ is selected from the group comprising hydrogen, a hetero C₃₋₇cycloalkyl, cyclopropyl and CF₃; and/or pharmaceutically acceptable salt or solvate thereof.
In a particular group of compounds of Formula I according to the invention, B is selected from the group comprising C₁-C₃alkyl optionally substituted with one or more fluoro atoms;
Z is selected from H or halogen; or B and Z together with the carbons to which they are attached form a 4-7 membered ring, optionally containing one or more heteroatoms, wherein the 4-7 membered ring optionally is substituted with one or more substituents selected from the group comprising C₁-C₃alkyl;
R₁ is selected from the group comprising heteroaryl and phenyl, optionally substituted with 1 or more halogen atoms;

In yet another aspect, the disclosure relates to a compound according to formula:

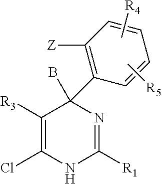

wherein B, Z, R₁ R₃, R₄, and R₅ are defined as above.

In another illustrative embodiment, compounds described in International Patent Publication No. WO2014/037480 can be used in accordance with the methods of the invention. For example, the present disclosure provides compounds, which can be represented by (i) novel compounds having the general formula I:

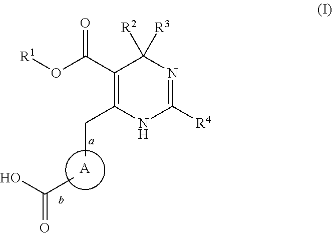

wherein
R¹ is C₁₋₆alkyl or trifluoromethyl-CₓH₂ₓ—, wherein x is 1-6;
one of R² and R³ is phenyl, which is once or twice or three times substituted by C₁₋₆alkyl, cyano or halogen; and the other one is hydrogen or deuterium;
R⁴ is phenyl, thiazolyl, oxazolyl, imidazolyl, thienyl or pyridinyl, which is unsubstituted or substituted by C₁₋₆alkyl, C₁₋₆alkylsulfanyl, halogen or cycloalkyl, where said C₁₋₆alkyl can be further optionally substituted with halogen;

### A is

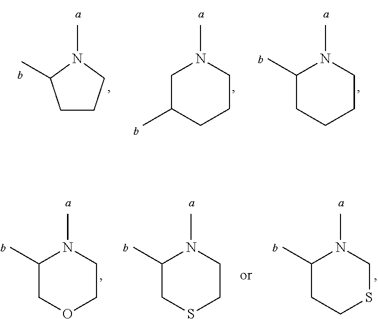

which is unsubstituted or substituted by groups selected from C₁₋₆alkyl, deuterium and halogen; or pharmaceutically acceptable salts, or enantiomers, or diastereomers thereof.

A further embodiment of the present disclosure is (ii) a compound of formula I, wherein

R¹ is methyl, ethyl, propyl, isopropyl, tert-butyl or trifluoromethylmethyl; one of R² and R³ is phenyl, which is once or twice or three times substituted by fluoro, chloro, bromo, iodo, methyl, or cyano; and the other one is hydrogen or deuterium;
R⁴ is phenyl, thiazolyl, oxazolyl, imidazolyl, thienyl or pyridinyl, which is unsubstituted or substituted by methyl, isopropyl, tert-butyl, bifluoromethyl, trifluoromethyl, cyclopropyl, methylsulfanyl, fluoro or chloro;

### A is

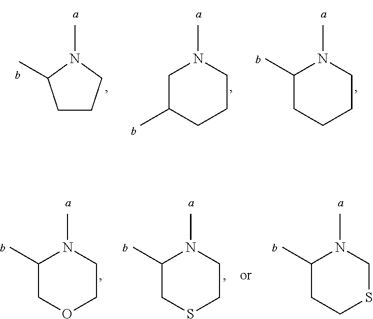

which is unsubstituted or substituted by groups selected from methyl, isopropyl, deuterium and fluoro; or pharmaceutically acceptable salts, or enantiomers, or diastereomers thereof.

Another embodiment of the present disclosure is (iii) a compound of formula I, wherein

R¹ is C₁₋₆alkyl or trifluoromethyl-CₓH₂ₓ—, wherein x is 1-6;
one of R² and R³ is phenyl, which is once or twice or three times substituted by C₁₋₆alkyl, cyano or halogen; and the other one is hydrogen or deuterium;
R⁴ is phenyl, thiazolyl, oxazolyl, imidazolyl, thienyl or pyridinyl, which is unsubstituted or substituted by C₁₋₆alkyl, C₁₋₆alkylsulfanyl, halogen or cycloalkyl, where said C₁₋₆alkyl can be further optionally substituted with halogen;

### A is

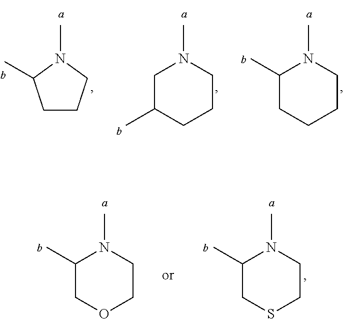

which is unsubstituted or substituted by groups selected from C₁₋₆alkyl, deuterium and halogen; or pharmaceutically acceptable salts, or enantiomers, or diastereomers thereof.

A further embodiment of the present disclosure is (iv) a compound of formula I, wherein

R¹ is methyl, ethyl, propyl, isopropyl or trifluoromethylmethyl;
one of R² and R³ is phenyl, which is once or twice or three times substituted by fluoro, chloro, bromo, iodo, methyl or cyano; and the other one is hydrogen or deuterium;
R⁴ is phenyl, thiazolyl, oxazolyl, imidazolyl, thienyl or pyridinyl, which is unsubstituted or substituted by methyl, isopropyl, trifluoromethyl, cyclopropyl, methylsulfanyl, fluoro or chloro;

### A is

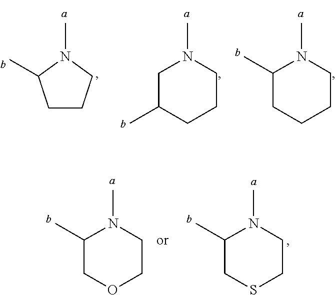

which is unsubstituted or substituted by groups selected from methyl, isopropyl, deuterium and fluoro; or pharmaceutically acceptable salts, or enantiomers, or diastereomers thereof.

Another embodiment of the present disclosure is (v) a compound of formula I, wherein

R¹ is C₁₋₆alkyl;
one of R² and R³ is phenyl, which is once or twice or three times substituted by C₁₋₆alkyl or halogen; and the other one is hydrogen or deuterium;
R⁴ is thiazolyl, oxazolyl, imidazolyl, thienyl or pyridinyl, which is unsubstituted or substituted by C₁₋₆alkyl, halogen or cycloalkyl, where said C₁₋₆alkyl can be further optionally substituted with halogen;

### A is

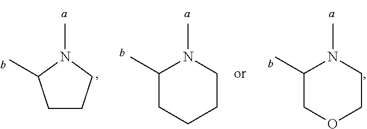

which is unsubstituted or substituted by groups selected from C₁₋₆alkyl, deuterium and halogen;
or pharmaceutically acceptable salts, or enantiomers, or diastereomers thereof.

A further embodiment of the present disclosure is (vi) a compound of formula I, wherein

R¹ is methyl or ethyl;
one of R² and R³ is phenyl, which is once or twice or three times substituted by fluoro, chloro, bromo, iodo or methyl; and the other one is hydrogen or deuterium;
R⁴ is thiazolyl, oxazolyl, imidazolyl, thienyl or pyridinyl, which is unsubstituted or substituted by methyl, isopropyl, trifluoromethyl, cyclopropyl or fluoro;

### A is

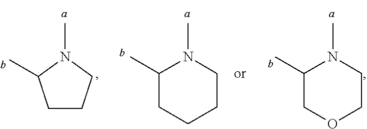

which is unsubstituted or substituted by groups selected from methyl, isopropyl, deuterium and fluoro;
or pharmaceutically acceptable salts, or enantiomers, or diastereomers thereof.

Another embodiment of the present disclosure is (vii) a compound of formula I or pharmaceutically acceptable salts, or enantiomers, or diastereomers thereof, wherein

R¹ is C₁₋₆alkyl;
one of R² and R³ is phenyl, which is once or twice or three times substituted by halogen; and the other one is hydrogen;

### R⁴ is

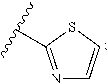 [SMILES: *c1nccs1]

### A is

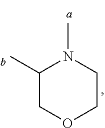

which is unsubstituted or substituted by C₁₋₆alkyl.

A further embodiment of the present disclosure is (viii) a compound of formula I or pharmaceutically acceptable salts, or enantiomers, or diastereomers thereof, wherein R¹ is methyl or ethyl;

one of R² and R³ is phenyl, which is once or twice or three times substituted by fluoro, chloro or bromo; and the other one is hydrogen;

### R⁴ is

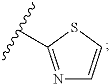 [SMILES: *c1nccs1]

### A is

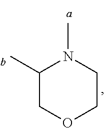

which is unsubstituted or substituted by methyl.

A further embodiment of the present disclosure is (ix) a compound of formula I or pharmaceutically acceptable salts, or enantiomers, or diastereomers thereof, wherein

R¹ is methyl or ethyl;
one of R² and R³ is

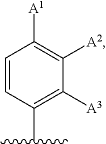

and the other one is hydrogen, wherein
A¹ is hydrogen or fluoro;
A² is hydrogen or fluoro;
A³ is fluoro, chloro or bromo; provided that at least one of A¹ and A² is hydrogen;

### R⁴ is

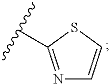 [SMILES: *c1nccs1]

### A is

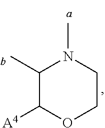

wherein A⁴ is hydrogen or methyl.

Another embodiment of the present disclosure is (x) a compound of formula I, wherein

R¹ is C₁₋₆alkyl or trifluoromethyl-CₓH₂ₓ—, wherein x is 1-6;
one of R² and R³ is phenyl, which is once or twice or three times substituted by C₁₋₆alkyl, cyano or halogen; and the other one is hydrogen or deuterium;
R⁴ is thiazolyl, oxazolyl, imidazolyl, thienyl or pyridinyl, which is unsubstituted or substituted by C₁₋₆alkyl, C₁₋₆alkylsulfanyl, halogen or cycloalkyl, where said C₁₋₆alkyl can be further optionally substituted with halogen;

### A is

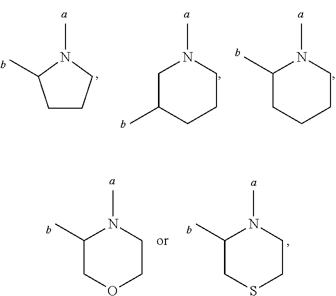

which is unsubstituted or substituted by groups selected from C₁₋₆alkyl, deuterium and halogen; or pharmaceutically acceptable salts, or enantiomers, or diastereomers thereof.

A further embodiment of the present disclosure is (xi) a compound of formula I, wherein

R¹ is methyl, ethyl, propyl, isopropyl or trifluoromethylmethyl;
one of R² and R³ is phenyl, which is once or twice or three times substituted by fluoro, chloro, bromo, methyl, or cyano; and the other one is hydrogen or deuterium;
R⁴ is thiazolyl, oxazolyl, imidazolyl, thienyl or pyridinyl, which is unsubstituted or substituted by methyl, isopropyl, trifluoromethyl, cyclopropyl, methylsulfanyl, fluoro or chloro;

### A is

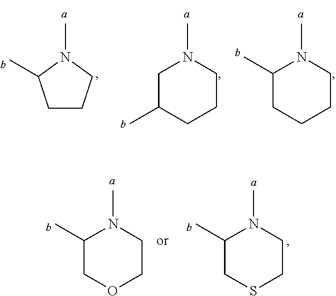

which is unsubstituted or substituted by groups selected from methyl, deuterium and fluoro;
or pharmaceutically acceptable salts, or enantiomers, or diastereomers thereof.

Another embodiment of the present disclosure is (xii) a compound of formula I, wherein

R¹ is C₁₋₆alkyl or trifluoromethyl-CₓH₂ₓ—, wherein x is 1-6;
one of R² and R³ is phenyl, which is once or twice or three times substituted by C₁₋₆alkyl or halogen; and the other one is hydrogen or deuterium;
R⁴ is thiazolyl, oxazolyl, imidazolyl, thienyl or pyridinyl, which is unsubstituted or substituted by C₁₋₆alkyl, C₁₋₆alkylsulfanyl, halogen or cycloalkyl, where said C₁₋₆alkyl can be further optionally substituted with halogen;

### A is

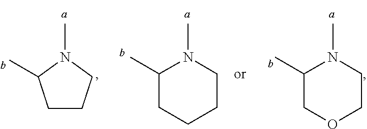

which is unsubstituted or substituted by groups selected from C₁₋₆alkyl, deuterium and halogen;
or pharmaceutically acceptable salts, or enantiomers, or diastereomers thereof.

A further embodiment of present disclosure is (xiii) a compound of formula I, wherein

R¹ is methyl, ethyl, isopropyl or trifluoromethylmethyl;
one of R² and R³ is phenyl, which is once or twice or three times substituted by fluoro, chloro, bromo, iodo or methyl; and the other one is hydrogen or deuterium;
R⁴ is thiazolyl, oxazolyl, imidazolyl, thienyl or pyridinyl, which is unsubstituted or substituted by methyl, isopropyl, trifluoromethyl, cyclopropyl, methylsulfanyl or fluoro;

### A is

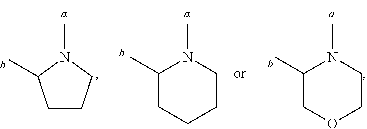

which is unsubstituted or substituted by groups selected from methyl, isopropyl, deuterium and fluoro;
or pharmaceutically acceptable salts, or enantiomers, or diastereomers thereof.

Another embodiment of the present disclosure is (xiv) a compound of formula I or pharmaceutically acceptable salts, or enantiomers, or diastereomers thereof, wherein

R¹ is C₁₋₆alkyl or trifluoromethyl-CₓH₂ₓ—, wherein x is 1-6;
one of R² and R³ is phenyl, which is once or twice or three times substituted by halogen; and
the other one is hydrogen or deuterium;

### R⁴ is

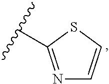 [SMILES: *c1nccs1]

which is unsubstituted or substituted by C₁₋₆alkyl, C₁₋₆alkylsulfanyl or cycloalkyl, where said C₁₋₆alkyl can be further optionally substituted with halogen;

### A is

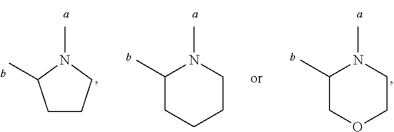

which is unsubstituted or substituted by groups selected from C₁₋₆alkyl, deuterium and halogen.

A further embodiment of the present disclosure is (xv) a compound of formula I or pharmaceutically acceptable salts, or enantiomers, or diastereomers thereof, wherein

R¹ is methyl, ethyl, propyl, isopropyl or trifluoromethylmethyl;
one of R² and R³ is phenyl, which is once or twice or three times substituted by fluoro, chloro, bromo or iodo; and the other one is hydrogen or deuterium;

### R⁴ is

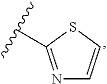 [SMILES: *c1nccs1]

which is unsubstituted or substituted by methyl, isopropyl, trifluoromethyl, cyclopropyl or methylsulfanyl;

### A is

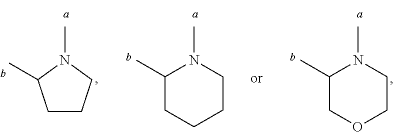

which may be unsubstituted or substituted by groups selected from methyl, isopropyl, deuterium and fluoro.

Another embodiment of the present disclosure is (xvi) a compound of formula I or pharmaceutically acceptable salts, or enantiomers, or diastereomers thereof, wherein

R¹ is C₁₋₆alkyl;
one of R² and R³ is

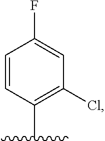 [SMILES: *c1ccc(F)cc1Cl]

and the other one is hydrogen;

### R⁴ is

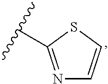 [SMILES: *c1nccs1]

which is unsubstituted or substituted by C₁₋₆alkyl;

### A is

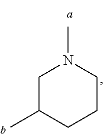

which is substituted by halogen.

Another embodiment of the present disclosure is (xvii) a compound of formula I or pharmaceutically acceptable salts, or enantiomers, or diastereomers thereof, wherein

R¹ is C₁₋₆alkyl;
one of R² and R³ is phenyl, which is substituted by halogen; and the other one is hydrogen;

### R⁴ is

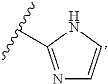 [SMILES: *c1ncc[nH]1]

which is substituted by C₁₋₆alkyl;

### A is

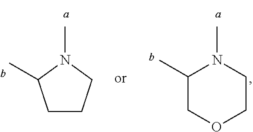

which may be unsubstituted or substituted by halogen.

Another embodiment of the present disclosure is (xviii) a compound of formula 1b or pharmaceutically acceptable salts, or enantiomers, or diastereomers thereof,

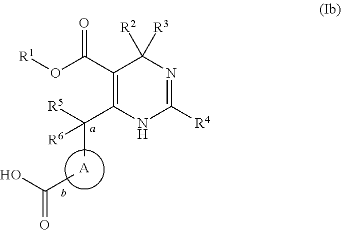

wherein
R¹ is C₁₋₆alkyl;
one of R² and R³ is phenyl, which is twice or thrice substituted by cyano or halogen; and the other one is hydrogen or deuterium;
R⁴ is phenyl, thiazolyl, oxazolyl, imidazolyl, thienyl or pyridinyl; which is unsubstituted or once or twice substituted by C₁₋₆alkyl, halogen, cycloalkyl or trifluoromethyl;
R⁵ is hydrogen;
R⁶ is hydrogen;

### A is

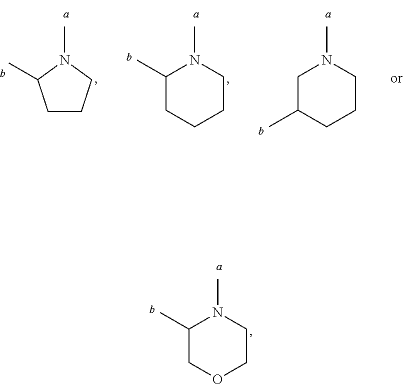

which may be unsubstituted or one, two, three or four times substituted by deuterium or halogen.

Additional compounds suitable for use with the methods described herein are found in Campana et al. (2013) J. Virol. 87(12):6931 and in International Patent Publication No. WO2013/006394, including, e.g., a compound having formula (I):

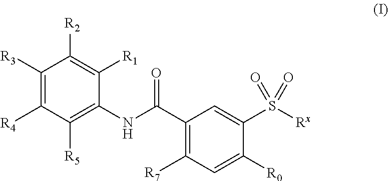

wherein
R¹ is hydrogen;
R² is selected from the group consisting of hydrogen, methyl, trifluoromethyl, fluorine, and chlorine;
R³ is selected from the group consisting of hydrogen, methyl, fluorine, and chlorine;
R⁴ is selected from the group consisting of hydrogen, fluorine, chlorine, and methyl;
R⁵ is selected from the group consisting of hydrogen and chlorine;
R⁷ is selected from the group consisting of hydrogen, chlorine, fluorine, and bromine;
R⁹ is selected from the group consisting of hydrogen, methyl, fluorine, and chlorine;
R⁹ is selected form a group consisting of NH₂,

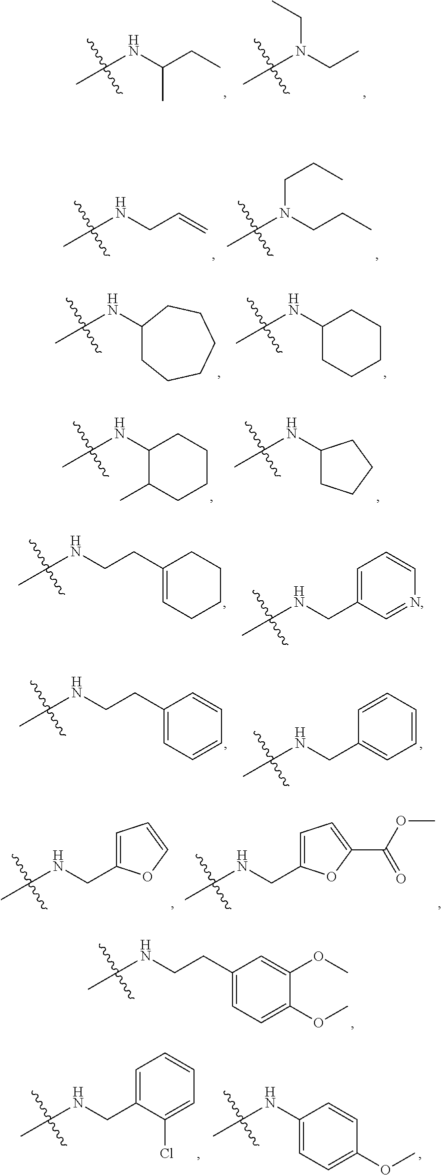

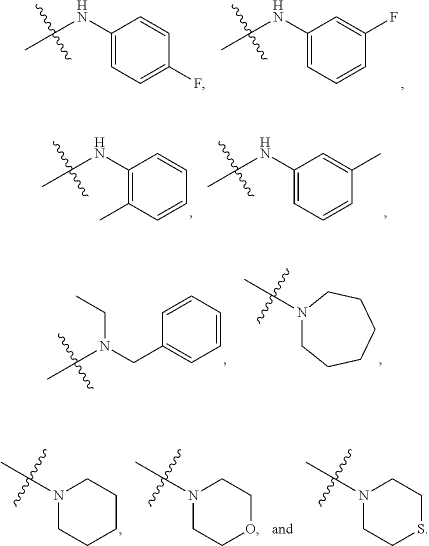

or a pharmaceutically acceptable salt form thereof.

Additional compounds of the disclosure are found in International Patent Publication No. WO2013/096744, including, e.g., a compound having formula (IV):

### Formula IV:

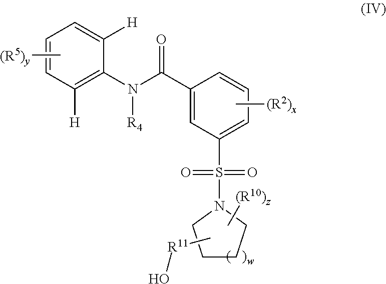

or pharmaceutically acceptable salts thereof wherein
R⁴ is H or C₁-C₃alkyl;
wherein each R⁵ is independently selected at each occurrence from the group consisting of CH₃, C₁-C₆alkoxy, halo, —CN, —NO₂, -(L)ₘ-SR⁹, -(L)ₘ-S(═O)R⁹, -(L)ₘ-S(═O)₂R⁹, -(L)ₘ-NHS(═O)₂R⁹, -(L)ₘ-C(═O)R⁹, -(L)ₘ-OC(═O)R⁹, -(L)ₘ-CO₂R⁸, -(L)ₘ-OCO₂R⁸, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆haloalkyl, —C₁-C₆dihaloalkyl and —C₁-C₆trihaloalkyl;
L is independently, at each occurrence, a bivalent radical selected from —(C₁-C₃alkylene)-, —(C₃-C₇cycloalkylene)-, —(C₁-C₃alkylene)ₘ-O—(C₁-C₃alkylene)ₘ-, or —(C₁-C₃alkylene)ₘ-NH—(C₁-C₃ alkylene)ₘ-;
each R⁸ is independently, at each occurrence, H, C₁-C₆alkyl, —C₁-C₆haloalkyl, —C₁-C₆dihaloalkyl, —C₁-C₆trihaloalkyl, C₁-C₆heteroalkyl, C₃-C₁₀cycloalkyl, C₃-C₁₀heterocycloalkyl, aryl, heteroaryl, —C₁-C₄alkyl-(C₃-C₁₀cycloalkyl), —C₁-C₄alkyl-(C₃-C₁₀heterocycloalkyl), —C₁-C₄alkyl-(aryl), or —C₁-C₄alkyl(heteroaryl), and wherein the alkyl, heteroalkyl, cycloalkyl, heterocycloalkyl, aryl and heteroaryl is optionally substituted with 1-5 substituents selected from R²;
R⁹ is —C₁₋₆alkyl, —C₁₋₆haloalkyl, —C₁₋₆dihaloalkyl, —C₁₋₆trihaloalkyl, C₁-C₆heteroalkyl, C₃-C₁₀cycloalkyl, C₃-C₁₀heterocycloalkyl, aryl, heteroaryl, —C₁-C₄alkyl-(C₃-C₁₀cycloalkyl), —C₁-C₄alkyl-(C₃-C₁₀heterocycloalkyl), —C₁-C₄alkyl-(aryl), or —C₁-C₄alkyl-(heteroaryl), and wherein the alkyl, heteroalkyl, cycloalkyl, heterocycloalkyl, aryl or heteroaryl ring is optionally substituted with 0-5 substituents selected from R²;
R¹⁰ is OH, —C₁₋₆alkyl, C₁₋₆alkyl —OH, —C₁₋₆haloalkyl, —C₁₋₆dihaloalkyl, —C₁₋₆trihaloalkyl, C₁₋₆alkyl heteroalkyl, C₃-C₁₀cycloalkyl, —C₃-C₁₀heterocycloalkyl, aryl, heteroaryl, —C₁-C₄alkyl-(C₃-C₁₀cycloalkyl), —C₁-C₄alkyl-(C₃-C₁₀heterocycloalkyl), —C₁-C₄alkyl-(aryl), or —C₁-C₄alkyl-(heteroaryl), and wherein the alkyl, heteroalkyl, cycloalkyl, heterocycloalkyl, aryl or heteroaryl ring is optionally substituted with 1-5 substituents selected from R²;
R¹¹ is a bond or C₁₋₃alkylene, wherein the C₁₋₃alkylene is optionally substituted with 1-3 substituents selected from R²;
R² is independently selected at each occurrence from the group consisting of halo, —CN, —NO₂, —C₁₋₆ alkyl, —C₁₋₆alkoxy, —C₁₋₆alkylhaloalkyl, —C₁₋₆dihaloalkyl, —C₁₋₆trihaloalkyl, —C₁₋₆heteroalkyl, and C(O)—C₁₋₆ alkyl;
w is 0, 1 or 2;
each occurrence of x is independently selected from the group consisting of 0, 1, 2, 3, and 4;
each occurrence of y is independently selected from the group consisting of 1, 2, and 3;
each occurrence of z is independently selected from the group consisting of 0, 1, 2, and 3;
each occurrence of m is independently 0, 1, or 2.

Additional compounds of the disclosure are found in International Patent Publication No. WO2014/033167, including, e.g., a compound of Formula (I)

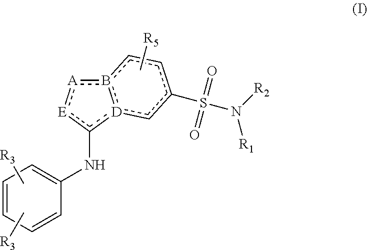

or a stereoisomer or tautomeric form thereof, wherein:
A represents N, C or O;
B represents C or N;
D represents C or N;
E represents C or N; wherein if A and E are either N or C, they are optionally substituted with R⁴;
R₁ represents hydrogen or C₁₋₃alkyl;
R₂ represents C₁₋₆alkyl, C₁₋₃alkyl-R₆, benzyl, or a 3-7 membered saturated ring optionally containing one or more heteroatoms each independently selected from the group consisting of O, S and N, such C₁₋₆alkyl or a 3-7 membered saturated ring optionally being substituted with one or more substituents each independently selected from the group consisting of hydrogen, halo, C₁₋₃alkyloxy, C₁₋₄alkyl, OH, CN, CFH₂, CF₂H or CF₃; or R₁ R₂ together with the nitrogen to which they are attached form a 5-7 membered saturated ring optionally being substituted with one or more substituents each independently selected from the group consisting of hydrogen, halogen, C₁₋₄alkyloxy, C₁₋₃alkyl, OH, CN, CFH₂, CF₂H and CF₃;
Each R₃ is independently selected from hydrogen, halo, C₁₋₄alkyloxy, C₁₋₄alkyl, OH, CN, CFH₂, CF₂H, CF₃ or a 3-5 membered saturated ring optionally containing one or more heteroatoms each independently selected from the group consisting of O and N;
R₄ represents hydrogen, C₁₋₄alkyl, C₃₋₅cycloalkyl, —(C═O)C₁₋₄-alkyl, —(C═O)—C₁₋₃alkyloxy or in case A or E equals C, R₄ in addition can be halogen;
R₅ represents hydrogen or halogen;
R₆ represents a 3-7 membered saturated ring optionally containing one or more heteroatoms each independently selected from the group consisting of O, S, and N, such 3-7 membered saturated ring optionally being substituted with one or more substituents each independently selected from the group consisting of hydrogen, halo, C₁₋₃alkyloxy, C₁₋₄alkyl, OH, CN, CFH₂, CF₂H, CF₃; or a pharmaceutically acceptable salt or a solvate thereof.

Additional compounds of the disclosure are found in International Patent Publication No. WO2014/033170, including, e.g., a compound of Formula (Ia)

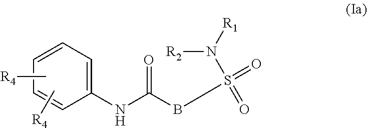

or a stereoisomer or tautomeric form thereof, wherein:
B represents a monocyclic 5 to 6 membered aromatic ring, optionally containing one or more heteroatoms each independently selected from the group consisting of O, S, and N, such 5 to 6 membered aromatic ring optionally being substituted with one or more substituents each independently selected from the group consisting of hydrogen, halo, —C₁-C₃alkyl, CN, CFH₂, CF₂H and CF₃;
R₁ represents hydrogen or —C₁₋₃alkyl;
R₂ represents —C₁₋₆alkyl, —C₁₋₃alkyl-R₅, benzyl, —C(═O)—R₅, CFH₂, CF₂H, CF₃, or a 3-7 membered saturated ring optionally containing one or more heteroatoms each independently selected from the group consisting of O, S, and N, such 3-7 membered saturated ring or C₁₋₆alkyl optionally being substituted with one or more substituents each independently selected from the group consisting of hydrogen, halo, —C₁₋₄alkyloxy, oxo, —C(═O)—C₁₋₃alkyl, —C₁₋₄alkyl, OH, CN, CFH₂, CF₂H and CF₃; or R₁ and R₂ together with the nitrogen to which they are attached form a 1,4-dioxa-8-azaspiro[4.5] moiety or a 5-7 membered saturated ring, optionally containing one or more additional heteroatoms each independently selected from the group consisting of O, S, and N, such 5-7 membered saturated ring optionally being substituted with one or more substituents each independently selected from the group consisting of hydrogen, halo, C₁₋₄alkyloxy, oxo, —C(═O)—C₁₋₃alkyl, —C₁₋₄alkyl, OH, CN, CFH₂, CF₂H and CF₃; each R₄ is independently selected from hydrogen, halo, —C₁₋₄alkyloxy, —C₁₋₄alkyl, OH, CN, CFH₂, CF₂H, CF₃ or a 3-5 membered saturated ring optionally containing one or more heteroatoms each independently selected from the group consisting of O and N;
R₅ represents —C₁₋₆alkyl, CFH₂, CF₂H, CF₃ or a 3-7 membered saturated ring optionally containing one or more heteroatoms each independently selected from the group consisting of O, S and N, such 3-7 membered saturated ring optionally being substituted with one or more substituents each independently selected from the group consisting of hydrogen, halo, C₁₋₄alkyloxy, oxo, —C(═O)—C₁₋₃alkyl, C₁₋₄alkyl, OH, CN, CFH₂, CF₂H and CF₃; or a pharmaceutically acceptable salt or a solvate thereof.

Additional compounds of the disclosure are found in International Patent Publication No. WO2014/033176, including, e.g., a compound of Formula I:

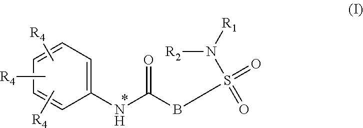

or a stereoisomer or tautomeric form thereof, wherein:
B represents a monocyclic 5 to 6 membered aromatic ring, optionally containing one or more heteroatoms each independently selected from the group consisting of O, S, and N, such 5 to 6 membered aromatic ring optionally being substituted with one or more substituents each independently selected from the group consisting of hydrogen, halogen, —C₁₋₃alkyl, CN, CFH₂, CF₂H and CF₃;
R₁ represents hydrogen or —C₁₋₃alkyl;
R₂ represents —C₁₋₆alkyl, —C₁₋₆alkenyl, —C₁₋₆alkyl-R₅, —C(═O)—R₅, CFH₂, CF₂H, CF₃, a dihydro-indenyl or tetrahydronaphthalenyl moiety optionally substituted with OH, or a 3-7 membered saturated ring optionally containing one or more heteroatoms each independently selected from the group consisting of O, S, and N, such 3-7 membered saturated ring, —C₁₋₆alkyl-R₅ or —C₁₋₆alkyl optionally being substituted with one or more substituents each independently selected from the group consisting of hydrogen, halogen, —C₁₋₄alkyloxy, —C₁₋₄alkyloxycarbonyl, oxo, —C(═O)—C₁₋₃alkyl, —C₁₋₄alkyl, OH, CN, CFH₂, CF₂H and CF₃; or R₁ and R₂ together with the nitrogen to which they are attached form a 6-10 membered bicyclic or bridged ring or a 5-7 membered saturated ring, such bicyclic, bridged or saturated ring moiety optionally containing one or more additional heteroatoms each independently selected from the group consisting of O, S, and N, such 5-7 membered saturated ring optionally being substituted with one or more substituents each independently selected from the group consisting of hydrogen, halogen, C₁₋₄alkyloxy, C₁₋₄alkyloxycarbonyl, oxo, C(═O)—C₁₋₃alkyl, —C₁₋₄alkyl, OH, CN, CFH₂, CF₂H and CF₃; each R₄ is independently selected from hydrogen, halo, C₁₋₄alkyloxy, C₁₋₄alkyl, C₁₋₄alkenyl, OH, CN, CFH₂, CF₂H, CF₃, HC≡C— or a 3-5 membered saturated ring optionally containing one or more heteroatoms each independently selected from the group consisting of 0 and N, such —C₁₋₄alkyl optionally substituted with OH;
R₅ represents —C₁₋₆alkyl, CFH₂, CF₂H, CF₃, phenyl, pyridyl or a 3-7 membered saturated ring optionally containing one or more heteroatoms each independently selected from the group consisting of O, S, and N, such 3-7 membered saturated ring optionally being substituted with one or more substituents each independently selected from the group consisting of hydrogen, halogen, —C₁₋₄alkyloxy, —C₁-C₄alkyloxycarbonyl, oxo, —C(═O)—C₁₋₃ alkyl, —C₁₋₄alkyl, OH, CN, CFH₂, CF₂H and CF₃;
or a pharmaceutically acceptable salt or a solvate thereof.

### Methods of Identifying Compounds

Also described herein are methods for identifying a compound useful for the treatment of infection by hepatitis B virus (HBV) and/or for clinically curing infection by HBV, comprising (a) measuring the ability of the compound to modulate core protein-mediated regulation of DNA (e.g., cccDNA) and (b) identifying the compound as useful for treating or clinically curing a hepatitis B infection based on the ability of the compound to modulate core protein-mediated regulation of DNA (e.g., cccDNA). The compound can modulate core protein-mediated regulation of DNA (e.g., cccDNA) by (a) modulating the structure of core protein; (b) modulating the function of core protein (thereby affecting, e.g., viral DNA levels, viral RNA levels, and/or viral antigen levels); (c) modulating the binding of core protein to DNA (e.g., cccDNA) (which can be assessed using, e.g., an EMSA assay); (d) depleting the amount of free core protein dimer available to bind to cccDNA; (e) altering nuclear import or export of core protein; (f) altering an interaction between DNA (e.g., cccDNA) and a chromatin component; (g) altering an interaction between core protein and a chromatin component; (h) altering the rate, quantity, quality or stability of RNA expressed from DNA (e.g., cccDNA); (i) altering the stability or maintenance of cccDNA; and/or (j) modulating an innate immune response against HBV. A compound useful for modulating an innate immune response against HBV can be assayed by measuring activation of APOBEC proteins (e.g., by PCT/sequencing of base pair changes secondary to APOBEC activity) or by assaying activation of a cytosolic DNA sensor, such as mitochondrial antiviral signaling protein (MAVS), DNA-dependent activator of IFN-regulatory factors (DAI), P202, LRRFIP1, or absent in melanoma 2 (AIM2), by evaluation of phosophostates and/or changes in downstream markers of immune activation.

The ability of the compound to modulate core protein-mediated regulation of DNA (e.g., cccDNA) can be measured by detecting a change in an amount of or state of core protein bound to DNA. Assays for detecting a change in the amount of core protein bound to DNA include chromatin immunoprecipitation (ChIP). Assays for detecting a change in the state of core protein bound to cccDNA include immunoprecipitation and mass spectrometry. A South-western blot of isolated DNA, e.g., cccDNA, can be performed using methods known in the art. Modulation of cccDNA can also be evaluated by a qPCR endpoint or real-time reporter assay in order to quantitatively assess either quantity of cccDNA compared to relaxed circular DNA (rcDNA), or to quantitatively assess production of viral RNAs. Other assays which can be used in the methods described herein include measuring viral antigen by ELISA and measuring viral RNA by qRT-PCR. Endpoint or real-time reporter assays can be used to detect changes in quantity of viral RNA (e.g., pgRNA) or protein production. Assays using energy transfer or quenching (1) between labeled core protein and DNA (e.g., cccDNA) or (2) between another DNA (e.g., cccDNA) binding protein and DNA can be used to show a compound's ability to, e.g., disrupt these binding interactions.

In some embodiments, the ability of the compound to modulate core protein-mediated regulation of DNA (e.g., cccDNA) also can be measured using assays known in the art to assess binding of the compound to a core protein dimer to determine whether the compound modulates core protein-DNA interaction (e.g., a core protein-cccDNA interaction). Differentially reporter-tagged core protein subunits can be used to assess binding of the compound to a core protein dimer. See, e.g., Example 3. In vitro binding of core protein to DNA (e.g., cccDNA) can also be measured. In one embodiment, binding is measure using a competition assay with control DNA. Methods of measuring the presence or quantity of a viral protein (e.g., HBsAg and HBeAg) or RNA (e.g., pgRNA) are known in the art. For example, clinical diagnostic kits for assessment of HBsAg and HBeAg are commercially available, e.g., from Roche® and Abbott®. In some embodiments, the ability of the compound to modulate core protein-mediated regulation of cccDNA is determined by differential scanning fluorimetry, isothermal calorimetry, thermopheresis, or Saturation Transfer Difference NMR. The method can also include varying the concentration of the compound until the compound modulates core protein-cccDNA interaction.

Methods for identifying a compound useful for the treatment of infection by hepatitis B virus (HBV) and/or for clinically curing infection by HBV, can include measuring the ability of the one or more compounds to modulate core protein structure or assembly and identifying the compound as useful for treating or clinically curing a hepatitis B infection based on the ability of the compound to modulate core protein. The method can also include measuring the ability of the compound to modulate core protein-mediated regulation of cccDNA.

Modulation of core protein activities can be measured by assessing binding of a labeled compound to a core protein dimer to determine whether the compound affects binding interactions of DNA (e.g., cccDNA) to the core protein dimer. Modulation of core protein assembly can also be determined by measuring fluorescence quenching of labeled core protein. See, e.g., Example 2. Modulation of core protein structure can be determined by measuring direct interaction with core protein, as with ITC or other methods known to those skilled in the art. The ability of the compound to modulate core protein can be measured by measuring altered binding of core protein to antibodies or other proteins sensitive to Cp tertiary or quaternary structure, immunoprecipitation and Western blot, sandwich ELISA, and/or a BRET assay.

### Therapy, Administration and Dosage

The term “therapeutically effective amount” as used herein, refers to that amount of active compound or pharmaceutical agent that elicits the biological or medicinal response in a tissue system, animal or human that is being sought by a researcher, veterinarian, nurse, pharmacist, physician assistant, medical doctor or other medical provider, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the medical provider within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, nurse, pharmacist, physician assistant, medical doctor or other medical provider of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a co-therapy.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

As used herein, the term “treatment” or “treating” means any administration of a compound or composition described and includes (1) inhibiting the disease in a patient that is experiencing or displaying the pathology or symptomatology of infection by HBV (i.e., arresting further development of the pathology and/or symptomatology), (2) ameliorating the disease in a patient that is experiencing or displaying the pathology or symptomatology of infection by HBV (i.e., reversing or lessening the pathology and/or symptomatology), inhibiting or (4) preventing of chronic infection by HBV. The term “controlling” includes preventing, treating, eradicating, ameliorating or otherwise reducing the severity of the infection by HBV, reducing production of new virions and/or prevention of hepatic inflammation.

As used herein, the term “curing” or “cure” means inactivation of cccDNA such that HBsAg and HBV DNA are produced at clinically insignificant levels. “Cure” or “curing” can also mean patients previously on therapy or requiring therapy are no longer deemed to require therapy for chronic HBV.

As used herein, the term “clinical outcome” means the manifestation of the disease in a patient that has experienced or displayed the pathology or symptomatology of infection by HBV after treatment. For example, a patient's clinical outcome can include inhibition or amelioration of the disease or symptoms of the disease or inhibiting or preventing of chronic infection or sequela of chronic infection by HBV.

The term “administering” as used herein includes all means of introducing the compounds and compositions described herein to the patient, including, but are not limited to, oral (po), intravenous (iv), intramuscular (im), subcutaneous (sc), transdermal, inhalation, and the like. The compounds and compositions described herein may be administered in unit dosage forms and/or formulations containing conventional nontoxic pharmaceutically-acceptable carriers, adjuvants, and vehicles.

Illustrative routes of oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

### EXAMPLES

### Example 1: Binding Labeled Compound to Cp or Capsid

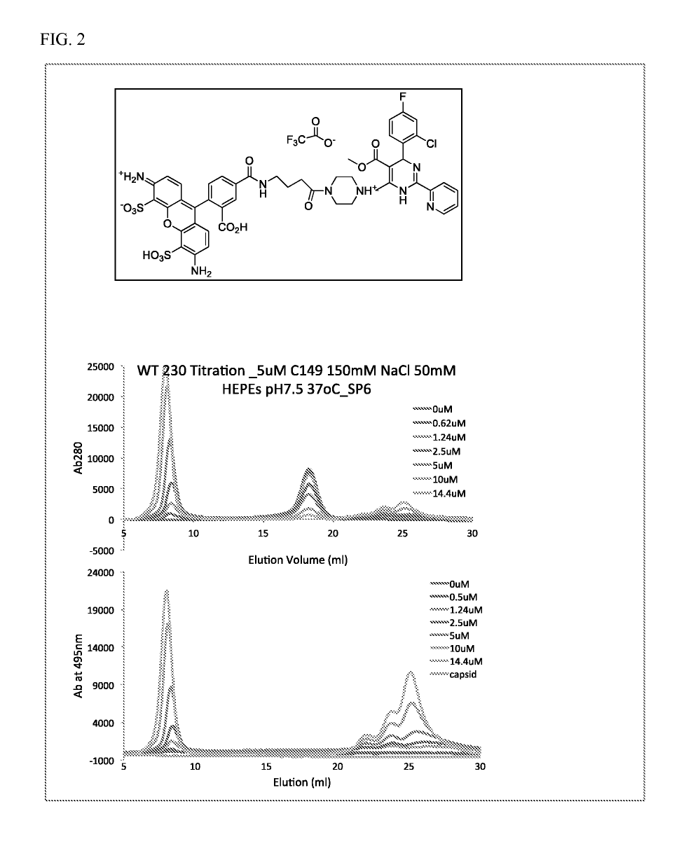

A fluorescent CpAM, such as FL-HAP (see FIG. 2), is added to a sample of Cp149 capsid, Cp149 dimer, or a mixture of the two as might be found in an assembly reaction. The compound is allowed to equilibrate. The small molecule in the method is to resolve capsid, dimer and free small molecule by size exclusion chromatography where the amount of labeled compound and protein in each fraction can be measured (see FIG. 2). (ii) Another method is measure changes in the fluorescence anisotropy of the small molecule when it binds to the much larger protein or protein complex. (iii) Yet another method is to look for changes in the fluorescence emission associated with the environment of the binding site compared to bulk solution. Another method to detect binding is to observe differences between free and bound fluorophore to the effect of a collisional quencher, such as iodide and acrylamide.

### Example 2: Measuring Assembly as a Means for Monitoring Depletion of Free Dimer

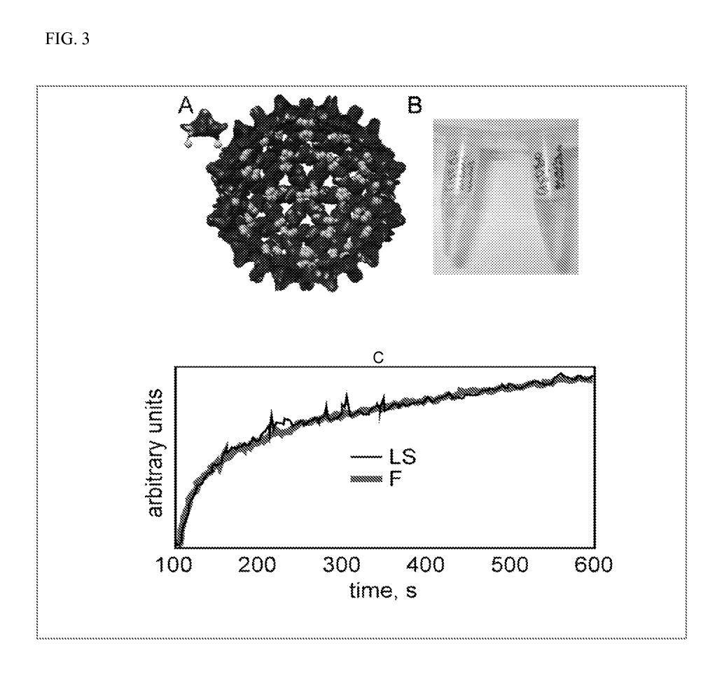

A high throughput fluorescence based assay is used to measure assembly as a means for monitoring depletion of free dimer. As shown in FIG. 3A, dimers have cysteines engineered at the C-termini, at either end, which is labeled with BoDIPY-FL, Cp150Bo. The free dimer is highly fluorescent. When Cp is induced to assemble by adjusting buffer conditions, as modulated by CpAMs, the fluorescent moieties are brought into close proximity at fivefold and quasi-sixfold vertices resulting in self-quenching of fluorescence. As shown in FIG. 3B, dimers are fluorescent (left) capsids (right) are not. FIG. 3C shows that the change in fluorescence matches other methods of monitoring assembly, such as light scattering (LS). Fluorescence monitoring has been used extensively to find molecules that drive assembly. Here the goal is to deplete the concentration of free Cp.

### Example 3: Assays Employing Differentially Reporter-Tagged Core Protein Subunits; In Vitro Binding of Core Protein to cccDNA; and In Vitro Binding of Labeled or Unlabeled Core Protein to Labeled or Unlabeled cccDNA in Competition with Unlabeled or Labeled Control DNA

In one form, unlabeled Cp is added to unlabeled HBV DNA and binding assessed by gel shift. Addition of a CpAM can differentially increase or weaken binding to viral DNA.

In a second form, Cp, fluorescently labeled, for example, by modification of cysteine 183 is used to titrate HBV DNA and the complex observed by native agarose gel shift.

In a third variation, the viral DNA is amplified by PCR using fluorescent oligomers. Binding to the labeled oligomer, especially in competition with non-specific unlabeled DNA can be measured by gel shift in a native agarose gel. CpAM addition will modify binding to labeled DNA.

Cp binding to HBV DNA and non-specific competitor DNA also can be measured in a high throughput manner. The HBV DNA is unlabeled and may be linear, as a component in a circular relaxed plasmid, or as a component on a circular supercoiled plasmid. The competitor DNA is an oligomer of 15 to 30 nucleotides whose sequence is only limited in that it not include specific HBV binding sites whether natural or synthetic. In this assay the Cp is labeled with a fluorophore, typically fluorescein. Also, a mutant Cp that carries the assembly-preventing Y132A mutant is used. There are two methods of read out. In the first method binding to cccDNA (1 MDa DNA or part of >2 MDa plasmid) is read by observing the anisotropy of the labeled Cp so that CpAM-induced loss of specificity is observed as a decrease in anisotropy. In the second method, the competitor DNA oligomers are labeled with a quencher so that CpAM-induced loss of specificity is read as a decrease in fluorescence.

### Example 4: Binding Assays Using Fluorescence

In another example, a fluorescently-labeled a structurally sensitive fluorophore is appended to a Cp-binding CpAM. The compound will exhibit enhanced fluorescence when bound to Cp conformations and substrates of interest. The bound compound can be displaced by a molecule competing for its ligand binding site or by a molecule binding to a different site that allosterically disrupts normal interactions at its binding site.

### Example 5: Size Exclusion Chromatography (SEC) and Anisotropy

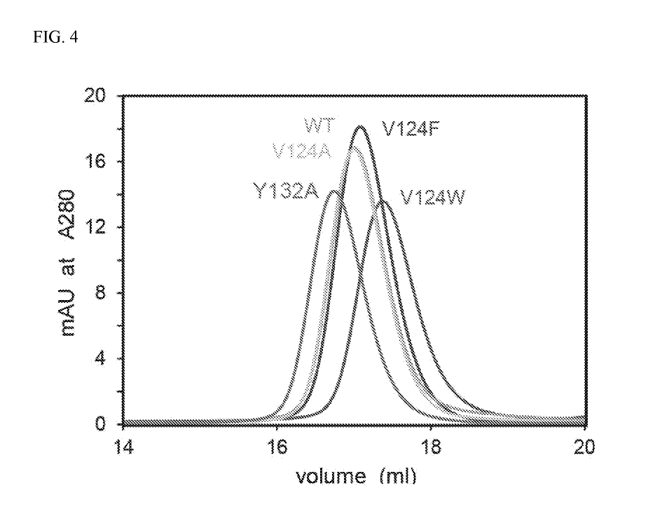

SEC can read out hydrodynamic radius (Stokes' radius) of a molecule as can anisotropy. Mutations of Cp that affect its in vitro activity (e.g. assembly) can appear as structural effects evident in SEC. Thus, simply measuring changes in SEC elution or change in anisotropy of a C-terminally labeled Cp signals both binding of Cp by a CpAM and a change in Cp structure. An exemplary SEC experiment is shown in FIG. 4.

### Example 6: Differential Scanning Fluorimetry (DSF)

Differential scanning fluorimetry (DSF) measures the temperature dependence of a protein's interaction with a fluorescent dye. Small molecules that bind to the target protein affect its stability. Using Sypro orange as the reporter dye we observe that HBV dimer undergoes two distinct dye-binding transitions at 66° C. and 74° C. Some dimer binding molecules have substantial concentration-dependent effects on the amplitude of the first transition. The concentration dependence provides a 66° C. KD value. The percent of the maximal amplitude change is reported as % ΔF. A few molecules also affect the second transition. These interactions demonstrate a drug-dependent change in the Cp-melting transition that correlates with limiting the structural transitions available to Cp.

### Example 7: Microscale Thermophoresis

Microscale thermophoresis measures binding of macromolecules (i.e. a protein) by relating binding to changes in diffusion of fluorescently-labeled molecule through a laser induced micro-temperature gradient. Moderate throughput of very small volume samples can be achieved. The instrument is sensitive to conformational changes and can thus be used with fluorescently labeled protein when the ligand modulates Stokes' radius. Thus the effects demonstrated in the SEC experiments will be readily evaluated. Of course, binding of a fluorescently-labeled small molecule to Cp 149 can easily be evaluated. Its displacement by unlabeled analogs that bind a competitive site is used to measure a dissociation constant.

### Example 8: Saturation Transfer Difference NMR (STD-NMR)

Saturation Transfer Difference NMR (STD-NMR) is a method for detecting ligands that are transiently bound to a protein. The protein resonances are saturated so that energy will be transferred to bound ligand, affecting the ligand NMR spectrum and allowing identification of parts of the ligand that are in contact with its receptor. Using solution conditions that inhibit assembly, ligand-Cp dimer interactions can be investigated.

### Example 9: Cell Based Assays

96-well plates (coated/white) are seeded with HBV producing cells in growth medium and incubated at 37° C. overnight. Test compound placed into wells as a single dose or as a series of dilutions. Media is replaced and new compounds added every 3 days. On day 9, cell viability/compound toxicity is measured and supernatant is assayed for viral load (genome equivalents) by qPCR, HBsAg and HBeAg (e.g., by ELISA).

This screening assay can also be performed with only one treatment day (in contrast to two treatment days as described above).

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

What is claimed is:

1. A method for treating or clinically curing a patient infected by hepatitis B virus (HBV), the method comprising administering to the patient a therapeutically effective amount of a first compound capable of modulating core protein-mediated regulation of HBV DNA in an HBV infected cell of the patient.

2. The method of claim 1, wherein the therapeutically effective amount of the first compound is capable of modulating core protein assembly.

3. The method of claim 1, the method further comprising administering to the patient a therapeutically effective amount of a second compound capable of modulating core protein assembly.

4. The method of claim 3, wherein administration of the first and second compounds to the patient results in improved clinical outcome compared to administration of either compound alone.

5. The method of claim 4, wherein administration of the first and second compounds results in a synergistically improved clinical outcome compared to administration of either compound alone.

6. A method for treating or clinically curing a patient infected by hepatitis B virus (HBV), the method comprising administering to the patient a therapeutically effective amount of a compound capable of modulating core protein assembly, wherein the compound is administered at a dose sufficient to modulate core protein-mediated regulation of HBV DNA.

7. The method of claim 6, wherein administration of the compound alters levels of at least one of HBsAg, HBeAg, and viral RNA.

8. The method of claim 1 wherein the therapeutically effective amount of the first compound, when administered to the patient, is capable of one or more of the following:
(a) modulating the structure of core protein;
(b) modulating the function of core protein;
(c) modulating the binding of core protein to HBV DNA;
(d) depleting the amount of free core protein dimer available to bind to HBV DNA;
(e) altering nuclear import or export of core protein;
(f) altering an interaction between HBV DNA and a chromatin component;
(g) altering an interaction between core protein and a chromatin component;
(h) altering the rate, quantity, quality or stability of RNA expressed from HBV DNA;
(i) altering the stability or maintenance of HBV DNA; and
(j) modulating an innate immune response against HBV.

9. The method of claim 1, wherein the first compound acts allosterically or orthosterically.

10. The method of claim 1, further comprising administering at least one additional compound selected from the group consisting of:
a nucleoside HBV polymerase inhibitor, a nucleotide HBV polymerase inhibitor, a modified nucleic acid, a peptide entry inhibitor, an interferon (Type I, II or III), a lymphotoxin beta agonist, a Toll-like receptor agonist, a non-nucleoside small molecule HBV polymerase inhibitor, a non-nucleotide small molecule HBV polymerase inhibitor, a compound capable of affecting capsid maturation, an HBV DNA transcriptional modulator, a DNA biosynthesis inhibitor, a subviral particle secretion inhibitor, a checkpoint modulator, an siRNA, a therapeutic vaccine, an entry inhibitor, a transcriptional modifier, a topoisomerase inhibitor, a compound that modulates presentation of HBV antigen via MHC, an HBV RNase H inhibitor, a proteasome inhibitor, a cyclophilin inhibitor, a transcription activator-like effector nuclease (TALEN), a DNA cleavage enzyme such as CAS9/CRISPR targeting HBV DNA, a dominant negative HBV mutant, a second mitochondrial-derived activator of caspases (SMAC) mimetic, a nucleic acid-based polymer (NAP) such as REP-2139Ca or REP-2055, a Stimulator of Interferon Genes (STING) such as DMXAA or 2′3′-cGAMP, and an inhibitory peptide.

11. The method of claim 10, wherein the checkpoint modulator is an anti-PD-1 antibody, an anti-PD-L1 antibody, or an anti-CTLA4 antibody.

12. The method of claim 10, wherein the siRNA targets HBV or host RNA.

13. The method of claim 1, wherein the first compound is:

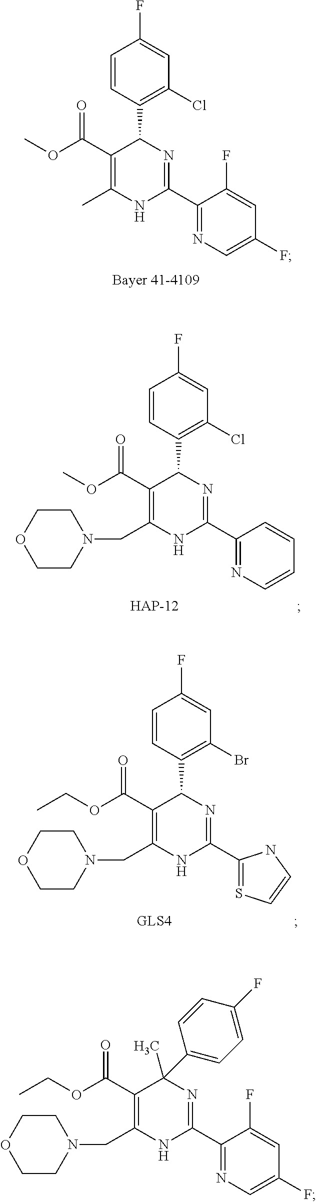

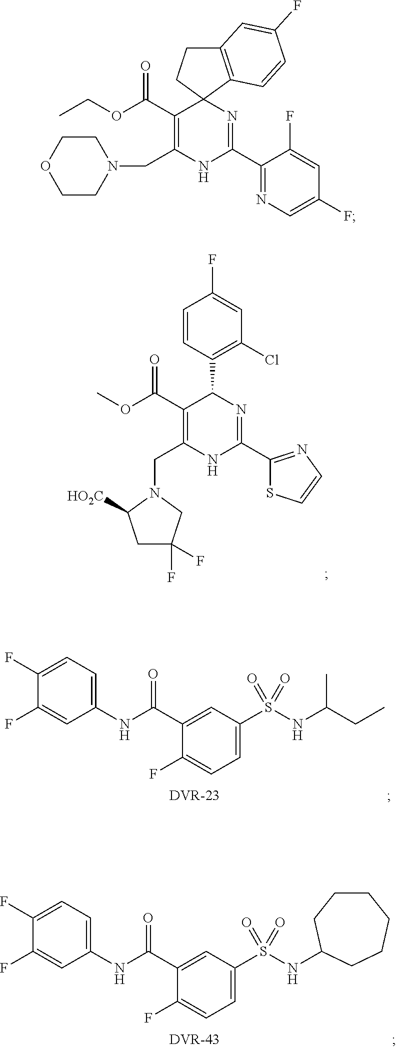

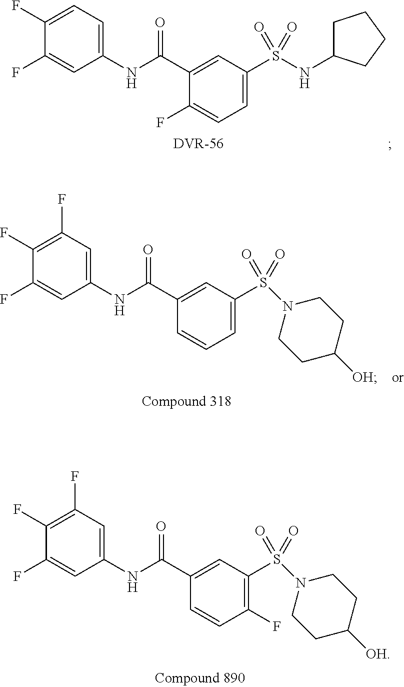

14. A method for identifying a compound useful for the treatment of infection by hepatitis B virus (HBV) and/or for clinically curing infection by HBV, comprising:
(a) measuring the ability of the compound to modulate core protein-mediated regulation of HBV DNA; and
(b) identifying the compound as useful for treating or clinically curing infection by HBV based on the ability of the compound to modulate core protein-mediated regulation of HBV DNA.

15. The method of claim 14, wherein measuring the ability of the compound to modulate core protein-mediated regulation of HBV DNA comprises measuring one or more of the following:
(a) modulation of the structure of core protein by the compound;
(b) modulation of the function of core protein by the compound;
(c) modulation of the binding of core protein to HBV DNA by the compound;
(d) depletion of the amount of free core protein dimer available to bind to HBV DNA by the compound;
(e) alteration of nuclear import or export of core protein by the compound;
(f) alteration of an interaction between HBV DNA and a chromatin component by the compound;
(g) alteration of an interaction between core protein and a chromatin component by the compound;
(h) alteration of the rate, quantity, quality or stability of RNA expressed from HBV DNA by the compound;
(i) alteration of the stability or maintenance of HBV DNA by the compound;
(j) modulation of an innate immune response against HBV by the compound;
(k) alteration of the encapsidation of pgRNA by the compound;
(l) recycling of HBV nucleic acid back to the nucleus to form HBV DNA; and
(m) production of host proteins whose genes are modulated by core protein, including interferon stimulated genes.

16. The method of claim 14, wherein measuring the ability of the compound to modulate core protein-mediated regulation of HBV DNA comprises one or more of the following:
(a) detecting a change in an amount of or state of core protein bound to HBV DNA, optionally by using a chromatin immunoprecipitation (ChIP) assay or immunoprecipitation and mass spectrometry;
(b) performing a South-western blot of isolated HBV DNA;
(c) evaluating isolated HBV DNA by a qPCR endpoint or real-time reporter assay;
(d) measuring viral antigen by ELISA;
(e) measuring viral RNA by qRT-PCR;
(f) performing an endpoint or real-time reporter assay;
(g) performing an assay using energy transfer or quenching between labeled core protein and HBV DNA or between another DNA binding protein and HBV DNA;
(h) performing surface plasmon resonance (SPR);
(i) performing biointerferometry;
(j) performing a fluorescence-based method such as FRET, FP, or fluorescence quenching; and
(k) detecting production of host proteins whose genes are modulated by core protein, including interferon stimulated genes.

17. The method of claim 14, wherein the ability of the compound to modulate core protein-mediated regulation of HBV DNA is measured by assessing binding of the compound to a core protein dimer to determine whether the compound affects binding interactions of HBV DNA to the core protein dimer.

18. The method of claim 14, wherein the ability of the compound to modulate core protein-mediated regulation of HBV DNA is measured using an assay comprising differentially reporter-tagged core protein subunits.

19. The method of claim 14, wherein the ability of the compound to modulate core protein-mediated regulation of HBV DNA is determined by measuring in vitro binding of core protein to HBV DNA, optionally comprising a competition assay with control DNA.

20. The method of claim 14, wherein the ability of the compound to modulate core protein-mediated regulation of HBV DNA is determined by measuring the presence or quantity of a viral protein or viral RNA.

21. The method of claim 20, wherein the method comprises measuring the presence or quantity of a HBsAg or HBeAg.

22. The method of claim 14, wherein the ability of the compound to modulate core protein-mediated regulation of HBV DNA is determined by an assay selected from the group consisting of:
differential scanning fluorimetry, isothermal calorimetry, thermopheresis, and Saturation Transfer Difference NMR.

23. The method of claim 14, further comprising varying the concentration of the compound until the compound modulates core protein-HBV DNA interaction.

24. A method for identifying a compound useful for the treatment of infection by hepatitis B virus (HBV) and/or for clinically curing infection by HBV, comprising:
(a) measuring the ability of the compound to modulate core protein assembly; and
(b) identifying the compound as useful for treating and/or clinically curing infection by HBV based on the ability of the compound to modulate core protein.

25. The method of claim 24, further comprising measuring the ability of the compound to modulate core protein-mediated regulation of HBV DNA.

26. The method of claim 25, wherein the ability of the compound to modulate core protein-mediated regulation of HBV DNA is measured by assessing binding of a labeled compound to a core protein dimer to determine whether the compound affects binding interactions of HBV DNA with the core protein dimer.

27. The method of claim 24, wherein the ability of the compound to modulate core protein assembly is determined by measuring fluorescence quenching of labeled core protein.

28. The method of claim 24, wherein the ability of the compound to modulate core protein assembly is measured by an assay selected from the group consisting of:
measuring altered binding of core protein to antibodies or other proteins sensitive to core protein tertiary or quaternary structure, immunoprecipitation and Western blot, sandwich ELISA, and a BRET assay.

29. The method of claim 1, wherein the HBV DNA is HBV cccDNA.
